SEQUENCE LISTING (1 sequence)

SEQ ID NO: 1 - protein, 29 aa, synthetic construct
  REGION 1..29: note = Synthetic Peptide
  MOD_RES 2: note = Aib
  SITE 16: note = Glu side chain lactam linkage to Lys
  SITE 16..20: note = cyclic
  CHAIN 17: note = Z17CO2H
  SITE 20: note = Lys side chain lactam linkage to Glu
HXQGTFTSDYSKYLDEKAAKEFIQWLLQT

(19) United States
(12) Patent Application Publication
(10) Pub. No.: US 2025/0288650 A1
(43) Pub. Date: Sep. 18, 2025

(54) Therapeutic Regimens and Methods for Reducing Body Weight and/or Serum Lipids using a GLP-1R and GCGR Agonist

(71) Applicant: Spitfire Pharma LLC, Gaithersburg, MD (US)

(72) Inventors: M. Scot Roberts, Bethesda, MD (US); Bertrand Victor Gilbert Georges, Stevenage (GB); Sarah K. Browne, Chevy Chase, MD (US); Matthew Scott Harris, Washington, DC, DC (US); John Joseph Suschak, III, Damascus, MD (US)

(73) Assignee: Spitfire Pharma LLC, Gaithersburg, MD (US)

(21) Appl. No.: 19/076,984

(22) Filed: Mar. 11, 2025

Related U.S. Application Data
(60) Provisional application No. 63/564,477, filed on Mar. 12, 2024.

(51) Int. Cl. A61K 38/26; A61K 47/18; A61K 47/26; A61P 3/04; A61P 3/06

(52) U.S. Cl. CPC A61K 38/26; A61K 47/183; A61K 47/26; A61P 3/04; A61P 3/06

(57) ABSTRACT

This disclosure relates to the once weekly dosing regimen of a dual GLP-1R and GCGR agonist, formulations, and methods of using the same for treatment of chronic weigh management and/or reducing serum lipids in a subject in need thereof with one or more co-morbidities using a pharmaceutical dosage formulation comprising a peptide product according to SEQ ID NO: 1, or peptide product that is at least 80% identical to a peptide of SEQ ID NO: 1.

### RELATED APPLICATIONS

This application claims priority to provisional application No. U.S. Ser. No. 63/564,477 filed 12 Mar. 2024 and which is hereby incorporated into this application in its entirety.

### SEQUENCE LISTING

The instant application contains a Sequence Listing which has been submitted electronically in ASCII format via the USPTO patent Center and hereby incorporated by reference in its entirety. Said ASCII copy, created on 6 Mar. 2025, is named MED014USPCT_ST26.xml and is 4.20 KB in size.

### FIELD OF THE DISCLOSURE

This disclosure relates to the use of the GLP-1R and GCGR agonist pemvidutide (a composition comprising SEQ ID NO.: 1) in certain dosing regimens for reducing body weight and/or serum pathogenic lipid mediators.

### BACKGROUND OF THE DISCLOSURE

Obesity is a major public health concern and a well-established risk factor for multiple metabolic and cardiovascular (CV) disorders. Obesity is a rapidly increasing problem worldwide and currently more than 65% of adults in the U.S. are overweight, and the number of obese people doubles yearly. Excess body weight is strongly associated with an increased risk of cardiovascular disease, hypertension, dyslipidemia, dysglycemia, diabetes, pre-diabetes, obstructive sleep apnea, osteoarthritis, and some cancers, contributing significantly to patient morbidity, mortality, and healthcare costs.

The increasing prevalence of cardiovascular (CV) disease and CV disease mortality in various human populations, including overweight and/or obese individuals, is a world health crisis of epidemic proportions that is a major contributor to patient morbidity and mortality as well as a major economic burden. For instance, obesity (e.g., persons having a body mass index (BMI, BMI kg/m²) of greater than 25 (overweight) or 30 (obese)) is a rapidly increasing problem worldwide and currently more than 65% of adults in the U.S. are overweight. And more than 80% of patients with heart failure with preserved ejection fraction (HFpEF) are overweight or obese (Kitzman, et al. Effect of caloric restriction or aerobic exercise training on peak oxygen consumption and quality of life in obese older patients with heart failure with preserved ejection fraction: A randomized clinical trial. JAMA. 2016; 315(1):36-46.) Weight loss of 7% has been shown to increase exercise tolerance and improve other measures of diastolic heart function in a study of 100 older patients (67+/−5 years) with obesity and clinically stable HFpEF (Id.; https://www.rethinkobesity.com/disease-progression/comorbidities-of-obesity.html). A majority of persons considered overweight or obese have limited options for available US Food and Drug Administration (FDA)-approved pharmacologic drugs for inducing weight loss, therapy has largely been based on lifestyle interventions directed at achieving weight loss. However, it is difficult to attain and maintain long-term weight loss with lifestyle changes alone.

Excess adiposity, particularly visceral fat, is a key driver of metabolic dysfunction and cardiovascular disease (CVD) in individuals with overweight or obesity. Adipose tissue is not merely a passive energy reservoir but an active endocrine organ that secretes inflammatory cytokines, free fatty acids, and adipokines that contribute to insulin resistance, dyslipidemia, and systemic inflammation. Elevated visceral fat is strongly associated with increased levels of low-density lipoprotein cholesterol (LDL-C), triglycerides (TGs), and non-high-density lipoprotein cholesterol (non-HDL-C)—all of which are major contributors to atherosclerosis and CVD. While weight reduction can improve lipid profiles, excessive loss of lean body mass during weight loss is undesirable, as lean body mass is essential for maintaining metabolic health, functional capacity, and resting energy expenditure. Preserving lean mass while preferentially reducing fat mass is crucial for optimizing metabolic outcomes, improving lipid metabolism, and sustaining long-term weight management. Pharmacologic interventions that not only facilitate fat loss but also preserve or enhance LBM may offer greater cardiometabolic benefits compared to conventional weight loss approaches.

Excess body weight is also frequently associated with dyslipidemia, characterized by elevated levels of total cholesterol, triglycerides (TGs), and low-density lipoprotein cholesterol (LDL-c). This dyslipidemic profile significantly increases the risk of atherosclerosis, coronary artery disease, and other cardiovascular complications. Elevated triglycerides and LDL-c contribute to plaque formation, endothelial dysfunction, and systemic inflammation, further exacerbating CV risk. Although weight loss alone can lead to modest lipid improvements, pharmacologic interventions that directly target both weight reduction and lipid abnormalities may provide superior benefits in reducing CV disease risk in overweight and obese individuals.

Glucagon-like peptide-1 receptor agonists (GLP-1RA) are associated with modest degrees of weight loss at approved doses, and these agents have emerged as a treatment option for patients that are overweight. GLP-1RAs exert central effects on appetite and food intake, while GCGR agonists (GCGRAs) drive increased energy expenditure and lipid oxidation in animal models and humans. The effects of GCGRAs and GLP-1RA have been shown to be synergistic in driving greater degrees of weight loss compared to a GLP-1RA alone. GCGRAs also enhance lipolysis and suppress liver fat synthesis, providing an additional pathway for obesity-related conditions such as liver fat reduction, and/or resolution or improvement of non-alcoholic fatty liver disease (NAFLD) or non-alcoholic steatohepatitis (NASH), both conditions associated with being overweight, and especially obese. However, it has not yet been shown that such agonists have any effect on CV risk factors such serum lipid levels or ratios.

Dual agonists combine GCGRAs with GLP-1RA in the same molecule. In obese non-human primates, chronic administration of a GLP-1R/GCGR dual agonist reduced body weight and improved glucose tolerance to a greater degree compared to a GLP-1RA mono-agonist. Clinical studies of cotadutide, a GLP-1/GCGR dual agonist with a 5:1 bias of GLP-1 to glucagon activity, demonstrated an impressive 39% reduction in liver fat content in just 6 weeks and greater improvement in NASH-related alanine aminotransferase (ALT) reduction than liraglutide alone. However, the degree of weight loss over 26 weeks of cotadutide administration was comparable to liraglutide (5.4% vs. 5.5%), suggesting that the 5:1 ratio was acceptable for liver fat reduction but suboptimal for weight reduction. Balanced (1:1) agonism has been shown to be associated with greater weight loss and metabolic effects than biased ratios that favor one agonist over the other. A recent study with JNJ 64565111, a balanced dual agonist, achieved an impressive 8% reduction in body weight in just 12 weeks (NCT03586830). It is unclear, however, whether the observed reduction in body weight has been associated with any CV disease-related factors (e.g., high cholesterol, triglycerides, LDL and VLDL particle concentration and/or diameter, and other related parameters).

Unfortunately, GLP-1Ras, and GLP-1R and GLP-1 based dual receptor agonists with bias towards GLP-1, have been associated with high rates of nausea, vomiting and diarrhea. These agents must also be titrated over prolonged periods to reduce side effects, and agents with improved tolerability and dosing regimens are needed.

Accordingly, there remains a need for convenient dosing (e.g., weekly instead of daily) with a therapeutic dose to reduce body weight and/or pathogenic plasma lipids in the prevention and/or treatment of metabolic disorders, including obesity, type 2 diabetes, CV disease-related conditions that in a therapeutic dose does not induce or exacerbate gastrointestinal side effects. The compounds and methods presented herein provide a solution to such problems, and in particular are shown to have beneficial effects on body weight and serum lipid levels which are both CV disease-related factors.

### SUMMARY OF THE DISCLOSURE

Described herein are methods for treatment of chronic weight management and/or reducing serum lipids. In embodiments provided herein are methods for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1, and wherein the human being in need thereof has a co-morbidity selected from one or more of cardiovascular disease, hypertension, dyslipidemia, dysglycemia, and obstructive sleep apnea. In certain embodiments, the treatment further reduces serum lipids.

In certain embodiments provided herein are methods for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1, and wherein the treatment further reduces serum lipids. In certain embodiments, the human being in need thereof has a co-morbidity selected from one or more of cardiovascular disease, hypertension, dyslipidemia, dysglycemia, and obstructive sleep apnea.

In certain embodiments, the peptide product is at least 85%, at least 90%, at least 95%, at least 96%, at least 97%, at least 98% or at least 99% identical to a peptide of SEQ ID NO: 1. In exemplary embodiments, the peptide product is 100% identical to a peptide of SEQ ID NO: 1.

In exemplary embodiments provided herein are pemvidutide formulations and uses of the same for inducing weight loss in a human being, the methods comprising administering to a human subject in need thereof a once weekly therapeutically effective dose of at least 1.2 mg and up to 2.4 mg of pemvidutide for at least 48 weeks. Also provided herein are pemvidutide formulations and uses of the same for reducing serum lipids in a human being, comprising administering to a human subject in need thereof a once weekly therapeutically effective dose of at least 1.2 mg and up to 2.4 mg of pemvidutide for at least 48 weeks. In some preferred embodiments, this disclosure provides a method for reducing body weight, comprising administering to a human subject in need thereof a once weekly therapeutically effective dose of at least 1.2 mg and up to 2.4 mg of pemvidutide for at least 48 weeks, optionally wherein the subject has a body mass index (BMI kg/m²) of greater than or equal to 25, is overweight wherein the subject has a BMI>27 kg/m², or is obese wherein the subject has a BMI>30 kg/m².

In embodiments provided herein are methods for treatment of reducing serum lipids in a human being in need thereof, comprising administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1. In certain embodiments, the human being in need thereof has a co-morbidity selected from one or more of obesity, cardiovascular disease, hypertension, dyslipidemia, dysglycemia, type 2 diabetes and obstructive sleep apnea.

In exemplary embodiments, the pharmaceutical dosage formulation comprises about 0.025-0.5% (w/w) polysorbate 20, about 0.2-0.5% (w/w) arginine, and about 3-6% (w/w) mannitol in water (pH 7.7±0.1). In certain embodiments, the pharmaceutical dosage formulation comprises about 0.2% (w/w) polysorbate 20, about 0.348% (w/w) arginine, about 4.260% (w/w) mannitol in water (pH 7.7±0.1).

In exemplary embodiments, the pharmaceutical dosage formulation is selected from one of a pharmaceutical formulation comprising a) (2.4 mg/ml; 0.24% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water; b) (1.8 mg/ml; 0.18% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water; or c) (1.2 mg/ml; 0.12% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Other embodiments are also contemplated as will be understood from the same by those of ordinary skill in the art.

### BRIEF DESCRIPTION OF THE DRAWINGS

FIG. 1 illustrates the disposition of the subjects in the Phase II Clinical Trial.

FIG. 2 illustrates the baseline characteristics of the subjects in the Phase II Clinical Trial.

FIG. 3 shows the relative weight loss achieved at week 48 at 2.4 mg pemvidutide. ***p<0.001 vs. placebo; mixed model for repeated measures (MMRM). See Example 3.

FIG. 4 shows the continuation of weight loss achieved at week 48 at 2.4 mg pemvidutide wherein a near linear trajectory of weight loss on 2.4 mg at 48 weeks is demonstrated.

FIG. 5 shows the absolute weight loss achieved at week 48 at 2.4 mg pemvidutide. *p<0.001 vs. placebo (MMRM). See Example 3.

FIG. 6 shows the majority of subjects lost ≥15% body weight at week 48. **p<0.005; ***p<0.001; ****p<0.0001 vs. placebo (CMH) Cochran-Mantel-Haenszel.

FIG. 7 shows robust weight loss at week 48 (placebo (FIG. 7A), 1.2 mg pemvidutide (FIG. 7B), 1.8 mg pemvidutide (FIG. 7C), and 2.4 mg pemvidutide (FIG. 7D)).

FIG. 8 shows significant reductions in BMI at week 48 compared to baseline for each of the three doses (1.2 mg, 18 mg and 2.4 mg). 49% of subjects on 2.4 mg realized a 1-class reduction in BMI; 29% of subjects on 2.4 mg realized a 2-class reduction in BMI; and, 48% of subjects on 2.4 mg with baseline obesity no longer had obesity at the end of treatment. Obesity class 1 (BMI 30-35); Obesity Class 2 (BMI 35-40); and Obesity Class 3 (BMI greater than 40).

FIG. 9 shows robust reductions in serum lipids (triglycerides; total cholesterol; LDL and VLDL) at week 48. *p<0.05; **p<0.005; ***p<0.001 vs. placebo (ANCOVA; analysis of covariance).

FIG. 10 shows greater reductions in triglycerides (FIG. 10A), total cholesterol (FIG. 10B), LDL cholesterol (FIG. 10C) at week 48 in subjects with elevated baseline levels. *p<0.05; **p<0.005; ***p<0.001 vs. placebo (ANCOVA). See Example 3.

FIG. 11 shows improvements in blood pressure (FIG. 11A) without clinically meaningful increases in heart rate (FIG. 11B) at week 48.

FIG. 12 shows glucose homeostasis (fasting glucose (FIG. 12A), HbA1c (FIG. 12B)) is maintained from baseline at week 48.

FIG. 13 provides an overview of adverse events (AEs).

FIG. 14 shows a ratio of lean weight loss to total weight loss (lean mass loss+adipose mass loss).

FIG. 15A shows a comparison of pemvidutide lean loss ratio and tirzepatide, retatrutide and semaglutide, wherein pemvidutide demonstrated only a 21.9% of weight loss to be from lean loss.

FIGS. 15B and 15C show subcutaneous fat and visceral fat loss for each of placebo, 1.2 mg dose, 1.8 mg dose and 2.4 mg dose of pemvidutide demonstrating a greater percent (preferential) loss of visceral fat (which is a risk factor for cardiovascular disease) compared to subcutaneous fat. See Example 4.

FIG. 16 shows a ratio of lean weight loss to total weight loss segmented by subjects age (less than 60 years old; N=38 (FIG. 16A) and 60 years or older; N=12 (FIG. 16B)) demonstrating a lower lean loss ratio for older subjects.

### DETAILED DESCRIPTION OF THE DISCLOSURE

This disclosure relates to methods for the treatment of chronic weigh management, including reducing body weight and/or maintaining weight loss, in a human being in need thereof comprising administering to the human being a once weekly therapeutic effective amount of a pharmaceutical dosage formulation comprising a peptide or peptide product of this disclosure. In embodiments, the human being in need thereof has a co-morbidity selected from one or more of cardiovascular disease, hypertension, dyslipidemia, dysglycemia, and obstructive sleep apnea. In certain embodiments, the treatment also reduces pathological serum lipids (e.g. LDL or VLDL). Accordingly, this disclosure also relates to methods for the treatment of reducing serum lipids in a human being in need thereof comprising administering to the human being a once weekly therapeutic effective amount of a pharmaceutical dosage formulation comprising a peptide or peptide product of this disclosure.

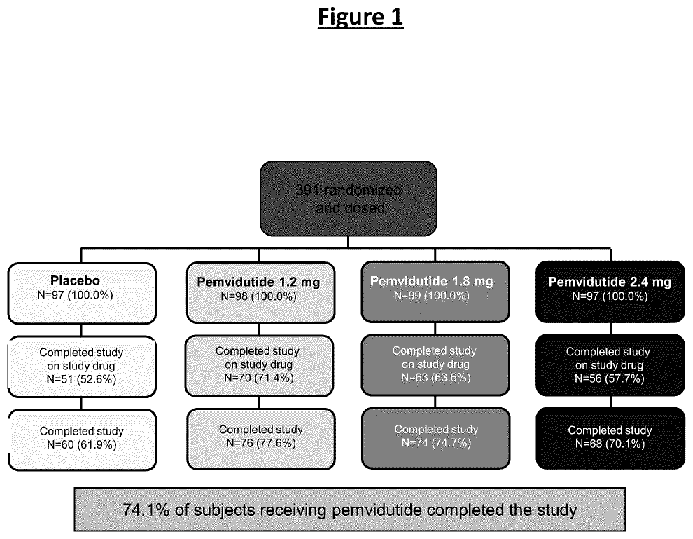

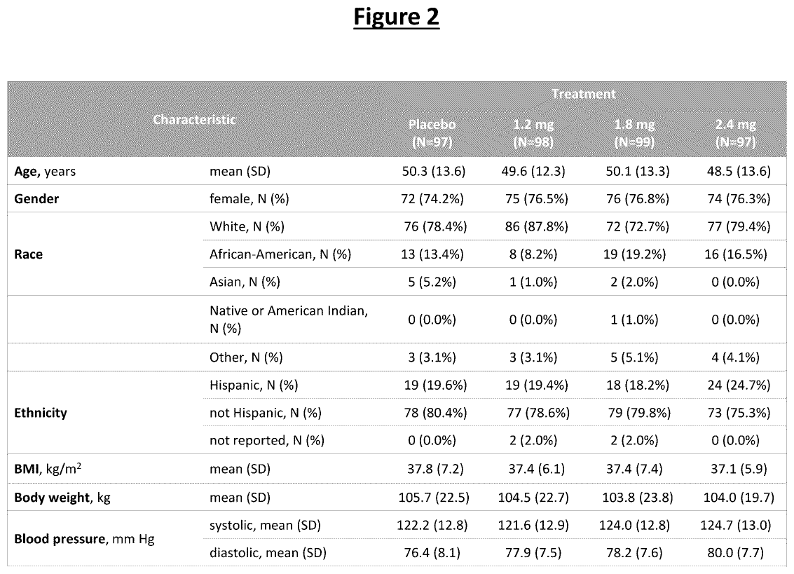

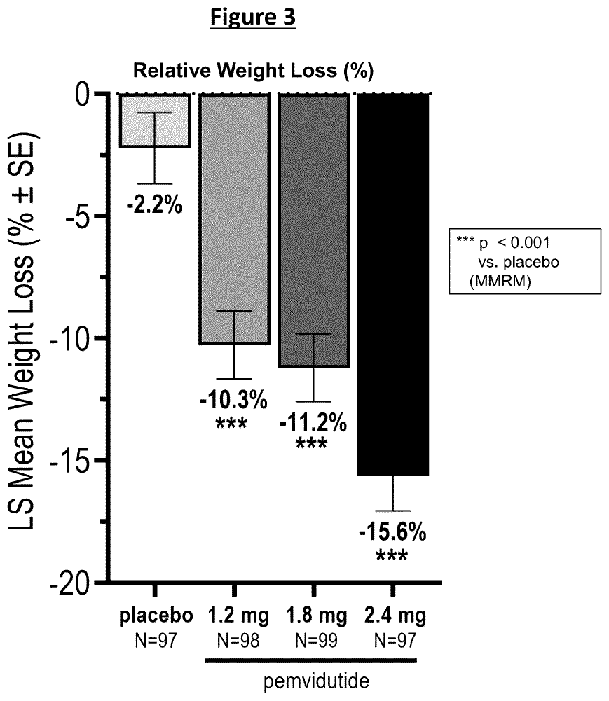

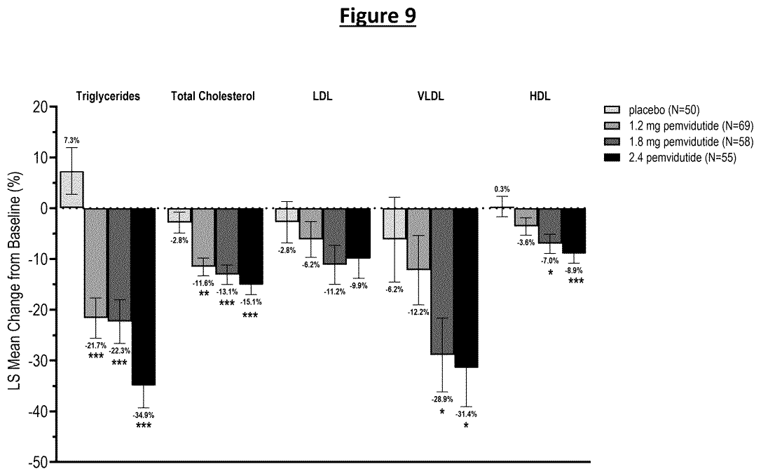

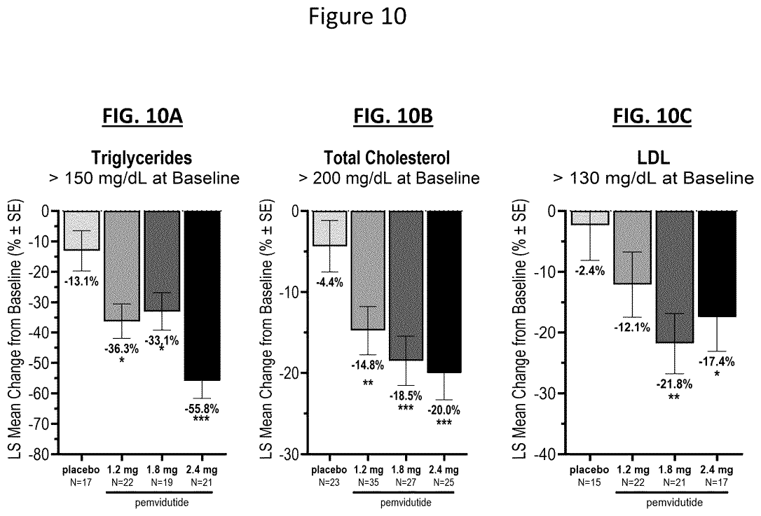

Applicant herein demonstrates that the peptide product of this disclosure (e.g., SEQ ID NO:1) reduces body weight comprising administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1. See Example 3 and FIGS. 3-8. The human beings in need thereof treated with a peptide product of this disclosure had a co-morbidity selected from one or more of cardiovascular disease, hypertension, dyslipidemia, dysglycemia, and obstructive sleep apnea. See FIGS. 1 and 2. Applicant herein further demonstrates that the peptide product of this disclosure (e.g., SEQ ID NO:1) reduces serum lipids comprising administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1. See Example 3 and FIGS. 9 and 10. Accordingly, provided herein are methods for the treatment of chronic weigh management using a peptide or peptide products of this disclosure, wherein the methods reduce body weight and/or serum lipids in a human being suffering from obesity or overweight, and with one or more co-morbidities (i.e., “a human being in need thereof”).

This disclosure relates to pemvidutide (a preferred “dual agonist peptide”) as well as pharmaceutical dosage formulations comprising, and methods for using, the same. Pemvidutide, as a dual agonist peptide, has affinity for, and in preferred embodiments about equal affinity for, glucagon-like peptide 1 receptor (GLP-1R) and glucagon receptor (GCGR), as may be determined using a cellular assay. In preferred embodiments, this disclosure provides methods for using pemvidutide (e.g., preferably pharmaceutical dosage formulation comprising SEQ ID NO: 1) that upon administration to a human being induces weight loss in the human being. In some embodiments, the human being is overweight, obese (e.g., a body mass index of at least about 25, with a range from at least 25 to at least 40), with one or more co-morbidities selected from one or more of cardiovascular disease, hypertension, dyslipidemia, dysglycemia, and obstructive sleep apnea. In preferred embodiments, the administration of pemvidutide as disclosed herein results in the weight loss and/or a reduction in waistline (“waist circumference”) measurements. In preferred embodiments, the weight loss and/or reduction in waistline measurement, is significant as measured against an untreated or control (e.g., vehicle-treated) individual and/or population. In some embodiments, the disclosure provides pharmaceutical dosage formulations configured to reduce pathogenic plasma lipid mediators. As used herein, “pathogenic” serum lipid mediators, included but are not limited to the following reactive lipid species: malondialdehyde (MDA), isolevuglandins (IsoLG), methyglyoxal (MGO), 4-oxononenal (ONE), and 4-hydroxynonenal (HNE). Oxidized phospholipids include 1-palmitoyl-2-oxovaleroyl-sn-glycero-3-phosphorylcholine (POVPC), 1-O-alkyl-2-azelaoyl-sn-glycero-3-phophorylcholine (azPAF), 1-(Palmitoyl)-2-(5-keto-6-octene-dioyl) phosphatidylcholine (KOdiA-PC), 1-palmitoyl-2-F2-isoprostane-sn-glycero-3-phosphocholine (F2IsoP-PC), and 1-palmitoyl-2-(5,6)-epoxyisoprostane E2-sn-glycero-3-phosphocholine (PEIPC). In certain embodiments, the pharmaceutical dosage formulations comprising pemvidutide are administered in a once weekly therapeutically effective dose to reduce serum lipids including total cholesterol, triglycerides, LDL, and/or VLDL.

In embodiments, the pharmaceutical dosage formulation disclosed herein comprises the peptide product of this disclosure, about 0.025-0.5% (w/w) polysorbate 20, about 0.2-0.5% (w/w) arginine, and about 3-6% (w/w) mannitol in water (pH 7.7±0.1). In exemplary embodiments, the pharmaceutical dosage formulation disclosed herein comprises SEQ ID NO: 1, about 0.2% (w/w) polysorbate 20, about 0.348% (w/w) arginine, about 4.260% (w/w) mannitol in water (pH 7.7±0.1).

In embodiments, the peptide product of this disclosure is administered once weekly in a therapeutically effective dose of at least 1.2 mg and up to 2.4 mg for about 48 weeks or more. In certain embodiments, a steady state therapeutic dose is achieved after a dose escalating phase having a duration of about 2 weeks, about 3 weeks or about 4 weeks, followed by administration through about 48 weeks. This is a preferred titration administered before the 2.4 mg once weekly dose.

In some embodiments, this disclosure provides pharmaceutical dosage formulations configured to reduce body weight including for treatment of chronic weight management and associated comorbidities. In some embodiments, the disclosure provides pharmaceutical dosage formulations configured to induce weight loss and reduce pathogenic serum lipid mediators for treatment of obesity (e.g. chromic weight management) and/or treatment for cardiovascular (CV) associated risk factors.

As used herein, the terms “treat,” “treated,” “treatment” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of chronic weight management” means an activity that alleviates or ameliorates any of the primary phenomena or secondary symptoms associated with the excess body weight (e.g., obesity or overweight) or other conditions described herein, including, but not limited to visceral fat, blood pressure and serum lipids. Thus, “treatment of (or for) reducing serum lipids” means an activity that alleviates or ameliorates any of the primary phenomena or secondary symptoms associated with elevated pathogenic serum lipids such as total cholesterol, triglycerides, LDL, and/or VLDL.

In certain embodiments provided herein are methods for reducing body weight (e.g., treatment of chronic weight management), comprising administering to a human subject in need thereof a once weekly therapeutically effective dose of a pharmaceutical dosage formulation comprising at least 1.2 mg and up to 2.4 mg of a peptide product according to SEQ ID NO: 1, or peptide product that is at least 80% identical to a peptide of SEQ ID NO: 1. In certain embodiments, the treatment is for at least 48 weeks. In certain embodiments, the human being in need thereof has a co-morbidity selected from one or more of cardiovascular disease, hypertension, dyslipidemia, dysglycemia, and obstructive sleep apnea. In certain other embodiments provided herein are methods for reducing serum lipids in a human being, comprising administering to a human subject in need thereof a once weekly therapeutically effective dose of a pharmaceutical dosage formulation comprising at least 1.2 mg and up to 2.4 mg of a peptide product according to SEQ ID NO: 1, or a peptide product that is at least 80% identical to a peptide of SEQ ID NO: 1. In certain embodiments, the peptide product is at least 85%, at least 90%, at least 95%, at least 96%, at least 97%, at least 98% or at least 99% identical to a peptide of SEQ ID NO: 1. In exemplary embodiments, the peptide product is 100% identical to a peptide of SEQ ID NO: 1 (i.e., pemvidutide).

Pemvidutide, present as a pharmaceutical formulation and comprising a synthetic peptide (SEQ ID NO: 1) is composed of naturally occurring amino acids and is a chimeric analog of the two native hormones GLP-1 and glucagon, with predominantly glucagon residues in the N-terminus and GLP-1 residues in the C-terminus. See U.S. Pat. No. 9,856,306, incorporated herein by reference. Pemvidutide also incorporates one nonproteinogenic amino acid, 2-aminoisobutyric acid, an amino acid side chain amide linkage (lactam bridge), and a surfactant side chain composed of a glucuronic acid linked to an octadecanoic fatty acid side chain. The surfactant side chain appears to slow entry in the circulation and may form micelles after subcutaneous (SC) injection. The lower maximal concentration (Cₘₐₓ) associated with slower entry may result in fewer GI side effects and better tolerability. This latter feature also enhances binding to plasma proteins and improves the metabolic stability, extending the half-life (t_{1/2}). The design of pemvidutide provides a co-agonist with equipotent (1:1) activity at both receptors of approximately 40 pM and 100% activity. Compositions comprising pemvidutide have been administered to human beings across a variety of doses and found not to induce side effects such as nausea, vomiting, diarrhea, abdominal pain and/or constipation using standard techniques. See US Patent Publ. No. 2021/0290732 and PCT Publication No. WO 2022/125598, each incorporated herein by reference.

SEQ ID NO: 1 has the following amino acid sequence:
¹His-²Aib-³Gln-⁴Gly-⁵Thr-⁶Phe-⁷Thr-⁸Ser-⁹Asp-¹⁰Tyr-¹¹Ser-¹²Lys-¹³Tyr-¹⁴Leu-¹⁵Asp-¹⁶Glu*-¹⁷Lys #-¹⁸Ala-¹⁹Ala-²⁰Lys*-²¹Glu-²²Phe-²³Ile-²⁴Gln-²⁵Trp-²⁶Leu-²⁷Leu-²⁸Gln-²⁹Thr-NH₂,
   where * indicates a lactam bridge is formed between Glu16 and Lys 20, and 17Lys # indicates the attachment site for glucuronic acid C-18-COOH (“1-(17-carboxylheptadecyloxy)-beta-D-glucuronyl”). Illustrated differently, SEQ ID NO: 1 is a peptide amide consisting of 29 amino acid residues and a glucuronic acid/Cis diacid moiety attached to ¹⁷Lys, in which the side-chains of ¹⁶Glu and ²⁰Lys form an intramolecular cycle as shown below:

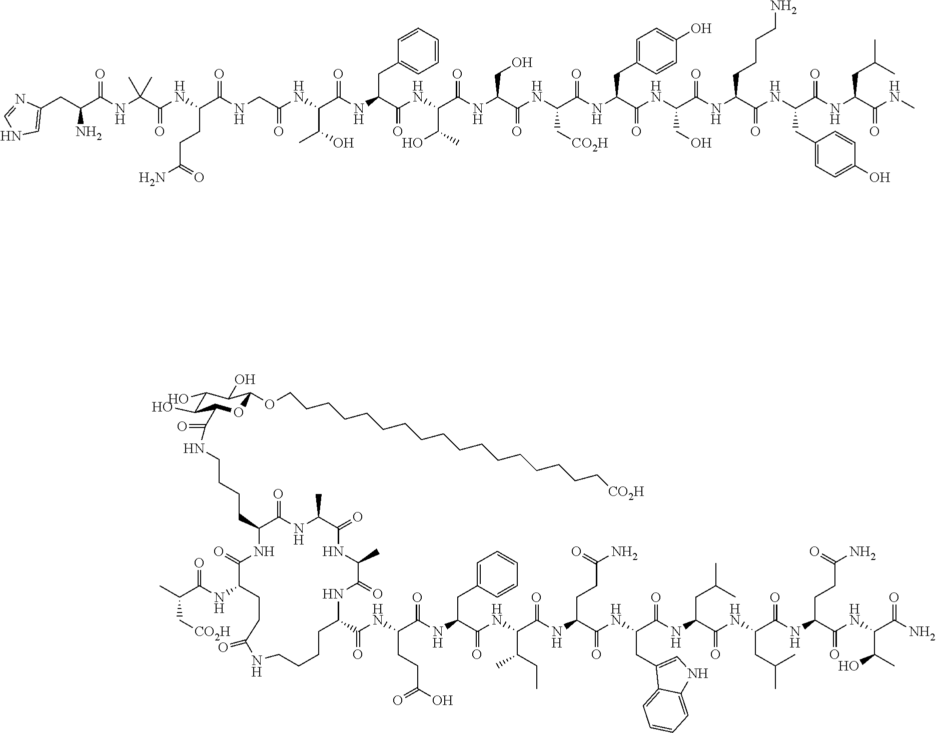

In embodiments provided herein is a pharmaceutical formulation of SEQ ID NO: 1 (referred to herein as pemvidutide) in an aqueous buffer solution. Pemvidutide, comprises an amino acid side chain amide linkage (lactam bridge), and a surfactant side chain composed of a glucuronic acid linked to a fatty acid side chain. The side chain, a surfactant comprised of a hydrophilic saccharide group covalently attached to the peptide via a linker amino acid, and a hydrophobic alkyl chain portion. The synthesis of SEQ ID NO. 1 is described in Example 1. In some embodiments, the dual agonist peptides can include one or more conservatively substituted amino acids as described herein. In preferred embodiments, SEQ ID NO: 1 can include one or more conservatively substituted amino acids, but preferably not at amino acid residues 16, 17, or 20 to provide a pemvidutide derivative. Provided herein is a peptide product that is 100% identical to SEQ ID NO: 1. In alternative embodiments, the peptide product is at least 85%, at least 90%, at least 95%, at least 96%, at least 97%, at least 98% or at least 99% identical to a peptide of SEQ ID NO: 1. In embodiments, the peptide product is at least 90% identical, at least 91% identical, at least 92% identical, at least 93% identical, at least 94% identical, at least 95% identical, at least 96%, at least 97%, at least 98% or at least 99% identical to a peptide of SEQ ID NO: 1. In embodiments, provided herein are methods wherein a peptide product that is at least 95% identical to SEQ ID NO: 1 is administered to a human being in need thereof in a pharmaceutical formulation of this disclosure.

A “peptide” (e.g., dual agonist peptide) comprises two or more natural or/and unnatural amino acid residues linked typically via peptide bonds. Such amino acids can include naturally occurring structural variants, naturally occurring non-proteinogenic amino acids, or/and synthetic non-naturally occurring analogs of natural amino acids. The terms “peptide” and “polypeptide” are used interchangeably herein. Peptides include short peptides (about 2-20 amino acids), medium-length peptides (about 21-50 amino acids) and long peptides (>about 50 amino acids, which can also be called “proteins”). In some embodiments, a peptide product comprises a surfactant moiety covalently and stably attached to a peptide of no more than about 50, 40 or 30 amino acids. Synthetic peptides can be synthesized using an automated peptide synthesizer, for example. Peptides can also be produced recombinantly in cells expressing nucleic acid sequences that encode the peptides. Conventional notation is used herein to portray peptide sequences: the left-hand end of a peptide sequence is the amino (N)-terminus, and the right-hand end of a peptide sequence is the carboxyl (C)-terminus. Standard one-letter and three-letter abbreviations for the common amino acids are used herein. Although the abbreviations used in the amino acid sequences disclosed herein represent L-amino acids unless otherwise designated as D- or DL- or the amino acid is achiral, the counterpart D-isomer generally can be used at any position (e.g., to resist proteolytic degradation). Abbreviations for other amino acids used herein include: Aib=a-aminoisobutyric acid (or 2-methylalanine or Ca-methylalanine); Xaa: any amino acid, typically specifically defined within a formula. Abbreviations for other amino acids that can be used as described herein include: Ac3c=1-aminocyclopropane-1-carboxylic acid; Ac4c=1-aminocyclobutane-1-carboxylic acid; Ac5c=1-aminocyclopentane-1-carboxylic acid; Ac6c=1-aminocyclohexane-1-carboxylic acid; Aib=alpha-aminoisobutyric acid (or 2-methylalanine or Calpha-methylalanine); Bip=3-(biphenyl-4-yl)alanine; Bip2Et=3-(2′-ethylbiphenyl-4-yl)alanine; Bip2EtMeO=3-(2′-ethyl-4′-methoxybiphenyl-4-yl)alanine; Cit=citrulline; Deg=2,2-diethylglycine; Dmt=(2,6-dimethyl)tyrosine; 2FPhe=(2-fluorophenyl)alanine; 2FMePhe or 2FaMePhe=Ca-methyl-(2-fluorophenyl)alanine; hArg=homoarginine; MeLys or aMeLys=Ca-methyllysine; MePhe or aMePhe=Ca-methylphenylalanine; MePro or aMePro=Ca-methylproline; Nall or Nal(1)=3-(1-naphthyl)alanine; Nal2 or Nal(2)=3-(2-naphthyl)alanine; Nle norleucine; Om=ornithine; and Tmp=(2,4,6-trimethylphenyl)alanine; 1,2,3,4-tetrahydroisoquinoline-3-carboxylic acid (Tic) and a Tic-Phe dipeptide moiety with a reduced amide bond between the residues (designated as Tic-Ψ[CF12-NFl]-Ψ-Phe) have the following structures:

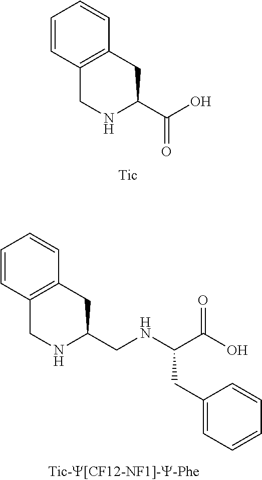 [SMILES: O=C(O)[C@@H]1Cc2ccccc2CN1 ; O=C(O)[C@H](Cc1ccccc1)NC[C@@H]1Cc2ccccc2CN1]

Unless specifically stated otherwise or the context clearly indicates otherwise, the disclosure encompasses any and all forms of a dual agonist peptide that may be produced, whether the dual agonist peptide is produced synthetically (e.g., using a peptide synthesizer) or by a cell (e.g., by recombinant production). Such forms of the dual agonist peptide (i.e., a pemvidutide derivative) can include one or more modifications that may be made during the course of synthetic or cellular production of the peptide, such as one or more post-translational modifications, whether or not the one or more modifications are deliberate. A dual agonist peptide can have the same type of modification at two or more different places, or/and can have two or more different types of modifications. Modifications that may be made during the course of synthetic or cellular production of a dual agonist peptide, including chemical and post-translational modifications, include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. A dual agonist peptide can have one or more modifications anywhere, such as the N-terminus, the C-terminus, one or more amino acid side chains, or the dual agonist peptide backbone, or any combination thereof. In some embodiments, a dual agonist peptide is acetylated at the N-terminus or/and has a carboxamide (—CONH₂) group at the C-terminus, which can increase the stability of the dual agonist peptide.

Potential modifications of SEQ ID NO: 1 can also include deletion of one or more amino acids, addition/insertion of one or more natural or/and unnatural amino acids, or substitution with one or more natural or/and unnatural amino acids, or any combination or all thereof. A substitution can be conservative or non-conservative. Such modifications may be deliberate, such as via site-directed mutagenesis or in the chemical synthesis of a dual agonist peptide, or may be accidental, such as via mutations arising in the host cell that produces the dual agonist peptide or via errors due to PCR amplification. An unnatural amino acid can have the same chemical structure as the counterpart natural amino acid but have the D stereochemistry, or it can have a different chemical structure and the D or L stereochemistry. Unnatural amino acids can be utilized, e.g., to promote a-helix formation or/and to increase the stability of the dual agonist peptide (e.g., to resist proteolytic degradation). A dual agonist peptide having one or more modifications relative to a reference dual agonist peptide may be called an “analog” or “variant” of the reference dual agonist peptide as appropriate. An “analog” typically retains one or more essential properties (e.g., receptor binding, activation of a receptor or enzyme, inhibition of a receptor or enzyme, or other biological activity) of the reference dual agonist peptide. A “variant” may or may not retain the biological activity of the reference dual agonist peptide, or/and may have a different biological activity. It is preferred that such a variant maintain its ability to act as an agonist of GLP-1R and GCGR, and in more preferred embodiments, has about equal affinity for GLP-1R and GCGR. In some embodiments, an analog or variant of a reference peptide has a different amino acid sequence than the reference dual agonist peptide.

The term “conservative substitution” refers to substitution of an amino acid in a dual agonist peptide with a functionally, structurally or chemically similar natural or unnatural amino acid. In certain embodiments, the following groups each contain natural amino acids that are conservative substitutions for one another: 1) Glycine (Gly/G), Alanine (Ala/A); 2) Isoleucine (Ile/I), Leucine (Leu/L), Methionine (Met/M), Valine (Val/V); 3) Phenylalanine (Phe/F), Tyrosine (Tyr/Y), Tryptophan (Trp/W); 4) Serine (Ser/S), Threonine (Thr/T), Cysteine (Cys/C); 5) Asparagine (Asn/N), Glutamine (Gln/Q); 6) Aspartic acid (Asp/D), Glutamic acid (Glu/E); and, 7) Arginine (Arg/R), Lysine (Lys/K), Histidine (His/H). In further embodiments, the following groups each contain natural amino acids that are conservative substitutions for one another: 1) non-polar: Ala, Val, Leu, Ile, Met, Pro (proline/P), Phe, Trp; 2) hydrophobic: Val, Leu, Ile, Phe, Trp; 3) aliphatic: Ala, Val, Leu, Ile; 4) aromatic: Phe, Tyr, Trp, His; 5) uncharged polar or hydrophilic: Gly, Ala, Pro, Ser, Thr, Cys, Asn, Gln, Tyr; 6) aliphatic hydroxyl- or sulfhydryl-containing: Ser, Thr, Cys; 7) amide-containing: Asn, Gln; 8) acidic: Asp, Glu; 9) basic: Lys, Arg, His; and, 10) small: Gly, Ala, Ser, Cys. In other embodiments, amino acids may be grouped as conservative substitutions as set out below: 1) hydrophobic: Val, Leu, Ile, Met, Phe, Trp; 2) aromatic: Phe, Tyr, Trp, His; 3) neutral hydrophilic: Gly, Ala, Pro, Ser, Thr, Cys, Asn, Gln; 4) acidic: Asp, Glu; 5) basic: Lys, Arg, His; and, 6) residues that influence backbone orientation: Pro.

Examples of unnatural or non-proteinogenic amino acids include without limitation alanine analogs (e.g., □-ethylGly [□-aminobutyric acid or Abu], □-n-propylGly [norvaline or Nva], □-tert-butylGly [Tbg], □-vinyl Gly [Vg or Vlg], □-allylGly [Alg], □-propargylGly [Prg], 3-cyclopropylAla [Cpa] and Aib), leucine analogs (e.g., nor-leucine, Nle), proline analogs (e.g., □-MePro), phenylalanine analogs (e.g., Phe(2-F), Phe(2-Me), Tmp, Bip, Bip(2′-Et-4′-OMe), Nal1, Nal2, Tic, □-MePhe, □-MePhe(2-F) and □-MePhe(2-Me)), tyrosine analogs (e.g., Dmt and □-MeTyr), serine analogs (e.g., homoserine [isothreonine or hSer]), glutamine analogs (e.g., Cit), arginine analogs (e.g., hArg, N,N′-g-dialkyl-hArg), lysine analogs (e.g, homolysine [hLys], Orn and □-MeLys), □,□□-disubstituted amino acids (e.g., Aib, □,□□-diethylGly [Deg], □-cyclohexylAla [2-Cha], Ac3c, Ac4c, Ac5c and Ac6c), and other unnatural amino acids disclosed in A. Santoprete et al., Pept. Sci., 17:270-280 (2011). □,□-Di-substituted amino acids can provide conformational restraint or/and a-helix stabilization. A reduced amide bond between two residues (as in, e.g., Tic-Ψ[CF12-NFl]-Ψ-Phe) increases protease resistance and may also, e.g., alter receptor binding. The disclosure encompasses all pharmaceutically acceptable salts of dual agonist peptides, including those with a positive net charge, those with a negative net charge, and those with no net charge.

In some embodiments, for instance, the dual agonist peptides can be associated with a saccharide, such as within a pharmaceutically acceptable composition or lyophilizate. Saccharides include monosaccharides, disaccharides and oligosaccharides (e.g., trisaccharides, tetrasaccharides and so on). A reducing saccharide exists in a ring form and an open-chain form in equilibrium, which generally favors the ring form. A functionalized saccharide of a surfactant moiety has a functional group suitable for forming a stable covalent bond with an amino acid of a dual agonist peptide.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition. In one embodiment, a pharmaceutically acceptable composition in which pemvidutide (or a derivative thereof) can be formulated comprises polysorbate 20 (e.g., about 0.2% (w/w)); optionally methylparaben (e.g., about 0.300% (w/w)); arginine (about 0.348% (w/w)), and mannitol (e.g., about 4.260% (w/w)) in distilled (DI) water.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician.

The disclosure also provides pharmaceutical compositions comprising a dual agonist peptide product described herein or a pharmaceutically acceptable salt thereof, and one or more pharmaceutically acceptable carriers or excipients. A pharmaceutical composition contains a therapeutically effective amount of a peptide product or an appropriate fraction thereof. A composition can optionally contain an additional therapeutic agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

In embodiments, a pharmaceutical formulation comprises a peptide product of this disclosure and about 0.025-0.5% (w/w) polysorbate 20, about 0.2-0.5% (w/w) arginine, about 3-6% (w/w) mannitol in water (e.g. deionized and/or sterilized) (pH 7.7±0.1); optionally about 0.2% (w/w) polysorbate 20, about 0.348% (w/w) arginine, about 4.260% (w/w) mannitol in water (pH 7.7±0.1). In certain embodiments, a present pharmaceutical formulation comprises SEQ ID NO: 1 (or a peptide product that is at least 80% identical to SEQ ID NO: 1) and about 0.2% (w/w) polysorbate 20, about 0.348% (w/w) arginine, about 4.260% (w/w) mannitol in water (pH 7.7±0.1). In certain embodiments, the pharmaceutical formulation comprises SEQ ID NO: 1 (or a peptide product that is at least 80% identical to SEQ ID NO: 1) and is configured for subcutaneous (SC) administration of a weekly therapeutic effective dose.

In certain embodiments, a peptide product of this disclosure is provided in a therapeutically effective dose of 1.2 mg/mL to 2.4 mg/mL. In certain embodiments the therapeutic formulation comprises a peptide product (2.4 mg/ml; 0.24% (w/w)); Mannitol (42.6 mg/mL; 4.26% (w/w)); Arginine (3.48 mg/mL; 0.348% (w/w)); and polysorbate 20 (2 mg/mL; 0.2% (w/w)) in sterile water. In certain embodiments the therapeutic formulation comprises peptide product (1.8 mg/ml; 0.18% (w/w)); Mannitol (42.6 mg/mL; 4.26% (w/w)); Arginine (3.48 mg/mL; 0.348% (w/w)); and polysorbate 20 (2 mg/mL; 0.2% (w/w)) in sterile water. In certain embodiments the therapeutic formulation comprises peptide product (1.2 mg/ml; 0.12% (w/w)); Mannitol (42.6 mg/mL; 4.26% (w/w)); Arginine (3.48 mg/mL; 0.348% (w/w)); and polysorbate 20 (2 mg/mL; 0.2% (w/w)) in sterile water. In embodiments, the peptide product is pemvidutide. In embodiments, the peptide product is SEQ ID NO: 1. In certain embodiments, the peptide product is at least 80%, at least 85%, at least 90% or at least 95% identical to SEQ ID NO: 1.

An appropriate or suitable formulation can depend on various factors, such as the route of administration chosen. Potential routes of administration of a pharmaceutical composition comprising a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository). In certain embodiments, a present dual agonist peptide product is administered parenterally (e.g., subcutaneously, intravenously or intramuscularly). In other embodiments, a peptide product is administered by oral inhalation or nasal inhalation or insufflation. In some embodiments, the carrier is an aqueous-based carrier, such as in a parenteral (e.g., subcutaneous, intravenous or intramuscular) formulation. In other embodiments, the carrier is a nonaqueous-based carrier. In certain embodiments, the nonaqueous-based carrier is a hydrofluoroalkane (HFA) or HFA-like solvent that may comprise sub-micron anhydrous a-lactose or/and other excipients, such as in a formulation for administration by oral inhalation or nasal inhalation or insufflation.

In some embodiments, a peptide product is administered parenterally (e.g., subcutaneously, intravenously or intramuscularly) by injection. Parenteral administration bypasses the strongly acidic environment of the stomach, gastrointestinal (GI) absorption and first-pass metabolism. Excipients and carriers that can be used to prepare parenteral formulations include without limitation solvents (e.g., aqueous solvents such as water, saline, physiological saline, buffered saline [e.g., PBS], balanced salt solutions [e.g., Ringer's BSS] and aqueous dextrose solutions), isotonic/iso-osmotic agents (e.g., salts [e.g., NaCl, KCl and CaCl₂] and sugars [e.g., sucrose]), buffering agents and pH adjusters (e.g., sodium dihydrogen phosphate [monobasic sodium phosphate]/di sodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Peptide formulations and delivery systems are discussed in, e.g., A. J. Banga, Therapeutic Peptides and Proteins: Formulation, Processing, and Delivery Systems, 3rd Ed., CRC Press (Boca Raton, Florida) (2015). The excipients can optionally include one or more substances that increase peptide stability, increase peptide solubility, inhibit peptide aggregation or reduce solution viscosity, or any combination or all thereof. Such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose}, osmolytes (e.g., trehalose, taurine, amino acids [e.g., glycine, sarcosine, alanine, proline, serine, b-alanine and g-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™F-68], and Genapol® PF-10 and variants thereof). Because such substances increase peptide solubility, they can be used to increase peptide concentration in a formulation. Higher peptide concentration in a formulation is particularly advantageous for subcutaneous administration, which has a limited volume of bolus administration (e.g., <about 1.5 mL). In addition, such substances can be used to stabilize peptides during the preparation, storage and reconstitution of lyophilized peptides. An exemplary parenteral formulation comprises a peptide product, mannitol, methionine, sodium thioglycolate, polysorbate 20, a pH adjuster (e.g., NaOH or/and HCl) and de-ionized water. Excipients of parenteral formulations that would be suitable for use with the dual agonist peptides described herein (e.g., various combinations of excipients including NaCl and the like) are well-known and available to those of ordinary skill in the art.

For parenteral (e.g., subcutaneous, intravenous or intramuscular) administration, a sterile solution or suspension of a peptide product in an aqueous solvent containing one or more excipients can be prepared beforehand and can be provided in, e.g., a pre-filled syringe of a single-use pen or a pen with a dose counter. Alternatively, a peptide product can be dissolved or suspended in an aqueous solvent that can optionally contain one or more excipients prior to lyophilization (freeze-drying). Shortly prior to parenteral administration, the lyophilized peptide product stored in a suitable container (e.g., a vial) can be reconstituted with, e.g., sterile water that can optionally contain one or more excipients.

A peptide product can also be administered by other routes, such as orally. An oral formulation can contain a peptide product and conventional excipients known in the art, and optionally an absorption enhancer such as sodium V-[8-(2-hydroxybenzoyl) aminocaprylate](SNAC). SNAC protects against enzymatic degradation via local buffering action and enhances GI absorption. An oral dosage form (e.g., a tablet, capsule or pill) can optionally have an enteric coating to protect its content from the strong acids and proteolytic enzymes of the stomach. In some embodiments, a peptide product is delivered from a sustained-release composition. As used herein, the term “sustained-release composition” encompasses sustained-release, prolonged-release, extended-release, delayed-release, slow-release and controlled-release compositions, systems and devices. In some embodiments, a sustained-release composition delivers a peptide product over a period of at least about 1 week, 2 weeks, 3 weeks, 1 month, 2 months, 3 months or longer. In further embodiments, a sustained-release composition is in the form of a depot that is generated when a mixture of a peptide product and a polymer is injected into a subject intramuscularly or subcutaneously. In certain embodiments, the polymer is or comprises PEG, polylactic acid (PLA) or polyglycolic acid (PGA), or a copolymer thereof (e.g., PLGA or PLA-PEG).

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug but can contain an appropriate fraction thereof so that taking multiple unit dosage forms achieves the therapeutically effective dose. Examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; and a capsule, cartridge or blister pre-loaded in or manually loaded into an inhaler. Alternatively, a pharmaceutical composition can be presented as a kit in which the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally). A kit can contain all active and inactive ingredients in unit dosage form or the active ingredient and inactive ingredients in two or more separate containers, and can contain instructions for administering or using the pharmaceutical composition to treat a medical condition disclosed herein. A kit can further contain a device for delivering the composition, such as an injection pen or an inhaler. In some embodiments, a kit contains a peptide product or a pharmaceutically acceptable salt thereof, or a pharmaceutical composition comprising the same, and instructions for administering or using the peptide product or the composition to treat a medical condition disclosed herein, such as insulin resistance, diabetes, metabolic syndrome, cardiovascular disease, obesity (including “chronic obesity” meaning obesity lasting more than one year or resulting in an obesity-related condition such as but not limited to insulin resistance, diabetes, metabolic syndrome, and/or cardiovascular disease), or a condition associated therewith (e.g.,). In certain embodiments, the kit further contains a device for delivering the peptide product or the composition, such as an injection pen.

### Methods of Treatment

Provided herein are methods for the treatment of chronic weight management comprising of a peptide product of this disclosure (e.g., SEQ ID NO: 1) to a subject in need thereof. In certain embodiments provided herein are methods for the treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1, and wherein the human being in need thereof has a co-morbidity selected from one or more of cardiovascular disease, hypertension, dyslipidemia, dysglycemia, and obstructive sleep apnea. In embodiments, the treatment methods reduce serum lipids. In embodiments, the human being in need thereof has aberrant serum lipid levels. In certain embodiments, the human being in need thereof has elevated levels of serum triglycerides, serum cholesterol, serum LDL and/or serum VLDL.

In certain embodiments provided herein are methods for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1, and wherein the treatment further reduces serum lipids. In embodiments, the human being in need thereof has a co-morbidity selected from one or more of cardiovascular disease, hypertension, dyslipidemia, dysglycemia, and obstructive sleep apnea. As used herein dyslipidemia is characterized by abnormally high (or low) amounts of one or more serum lipids. In embodiments, dyslipidemia is selected from the group consisting of hypercholesterolemia, hypertriglyceridemia, high/low density lipoprotein cholesterolemia, high non-high density lipoprotein cholesterolemia and low/high density lipoprotein cholesterolemia

The term “cardiovascular-related disease” herein refers to any disease or disorder of the heart or blood vessels (i.e. arteries and veins) or any symptom thereof, or any disease or condition that causes or contributes to a cardiovascular disease. Non-limiting examples of cardiovascular-related diseases and disorders include acute cardiac ischemic events, acute myocardial infarction, angina, angina pectoris, arrhythmia, atrial fibrulation, atherosclerosis, arterial fibrillation, cardiac insufficiency, cardiovascular disease, chronic heart failure, chronic stable angina, congestive heart failure, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hyperlipidemia in HIV positive subjects, hypertension, hypertriglyceridemia, ischemic complications in unstable angina and myocardial infarction, low blood pressure, metabolic syndrome, mixed dyslipidemia, moderate to mild heart failure, myocardial infarction, obesity management, paroxysmal atrial/arterial fibrillation/fibrulation/flutter, paroxysmal supraventricular tachycardias (PSVT), particularly severe or rapid onset edema, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitisterolemia, stroke, supraventricular tachycardia, symptomatic atrial fibrillation/flutter, tachycardia, type-II diabetes, vascular disease, venous thromboembolism, ventricular arrhythmias, and other cardiovascular events.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. The term “treating” as used herein includes prophylaxis of the named condition or amelioration or elimination of the condition once it has been established. The term “obesity” as used herein refers to a disease, medical condition or disorder and is defined as a body mass index (BMI)>30. A BMI of 25-30 is pre-obese (also referred to herein as “overweight”). As used herein methods for treating chronic weigh management refer to treatment of obesity and pre-obese.

Administration of an “effective amount” of the formulations and peptide products described herein is defined as an amount effective at dosages and for periods of time necessary to achieve the desired result. In one embodiment, the compositions may be administered or used weekly to provide a therapeutic effective dose in the treatment of chronic weight management and further reduce serum lipids and/or treat obesity related co-morbidities.

According to the present invention, to regulate (control) body weight can refer to reducing or increasing body weight, reducing or increasing the rate of weight gain, or increasing or reducing the rate of weight loss, and also includes actively maintaining, or not significantly changing body weight (e.g., against external or internal influences which may otherwise increase or decrease body weight) (“treatment of chronic weigh management”). One embodiment of the present invention relates to regulating body weight by administering to a human in need thereof a pharmaceutical formulation comprising a peptide product of this disclosure.

In certain embodiments, the present invention relates to methods and formulations for reducing fat mass and/or reducing gain of fat mass in a human being, and more particularly, for treating or ameliorating obesity in patients at risk for or suffering from obesity or overweight. Such methods are effective to reduce weight and/or mass loss, or to improve conditions associated with excess body weight and/or aberrant lipid levels.

In certain embodiments, the human in need thereof has a disease or disorder characterized by increased cholesterol accumulation in serum and/or tissue causing pathology or having excessive cholesterol as a risk factor. Cholesterol disorders include familial hypercholesterolemia or inherited cholesterol disorder (ICD), defects in the gene products of cholesterol metabolism e.g. 7-alpha-hydroxylase, and various forms of xanthomas. Increased levels of serum cholesterol may indicate atherosclerosis, biliary cirrhosis, familial hyperlipidemias, high-cholesterol diet, hypothyroidism, myocardial infarction, nephritic syndrome and uncontrolled diabetes. “Excessive cholesterol” means outside the typical (normal) cholesterol range. Typical cholesterol level is less than 200 mg/dL. Borderline High is 200-239 mg/dL and anything over 240 mg/dL is high.

There are two types of cholesterol, HDL cholesterol (sometimes called good cholesterol) and LDL cholesterol (sometimes called bad cholesterol). “Excessive cholesterol” may also be determined with respect to LDL. For example, treatment of a human being in need thereof is provided when LDL cholesterol is: >100 mg/dL (eg. with a goal to reduce LDL cholesterol to <100 mg/dL), at least 130 mg/dL (eg. with a goal to reduce LDL cholesterol to less than 130 mg/dL), at least 160 mg/dL (eg. with a goal to reduce LDL cholesterol to less than 130 mg/dL), or at least 190 mg/dL (eg. with a goal to reduce LDL cholesterol to less than 160 mg/dL).

In certain embodiments, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the human in need thereof exhibits one or more of reduced body weight, maintenance of body weight, reduction in serum lipid levels (e.g., cholesterol, LDL, VLDL or triglycerides), reduction in blood pressure and/or reduction in one more cardiovascular disease symptoms.

As detailed in Example 3, weekly treatment with a peptide product of this disclosure (e.g. SEQ ID NO: 1) yielded statistically significant reductions in bodyweight in a dose-dependent manner, with participants in the 2-4 mg cohort losing an average of 15.6%. Without wishing to be bound by a theory, the addition of glucagon to GLP-1 may provide for additional weight loss compared to GLP-1R agonists alone (e.g., semaglutide) due to increased energy expenditure. The ongoing weight loss at Week 48 suggests that the addition of glucagon may have readjusted the metabolic set point. Bodyweight reductions of 15% or more can improve many obesity related co-morbidities, including reversal of T2DM and resolution of MASH. More than 50% of participants in the 2·4 mg pemvidutide cohort lost ≥15% and 32% of participants lost; ≥20% of their bodyweight. Other co-morbidities with positive outcomes when reduced or ameliorated include cardiovascular disease, hypertension, dyslipidemia, dysglycemia, and obstructive sleep apnea. Waist circumference, and by extension visceral adiposity, is an indicator of risk for cardiovascular disease. Reductions in 5-10 cm correlate with improved serum lipid profiles and decreased diastolic blood pressure. All pemvidutide dose cohorts had mean reductions >6 cm, suggesting the possibility of improvements in these cardiovascular risk factors. Accordingly, provided herein are methods for the treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1, and wherein the method also treats one or more of cardiovascular disease, hypertension, dyslipidemia, dysglycemia, and/or obstructive sleep apnea.

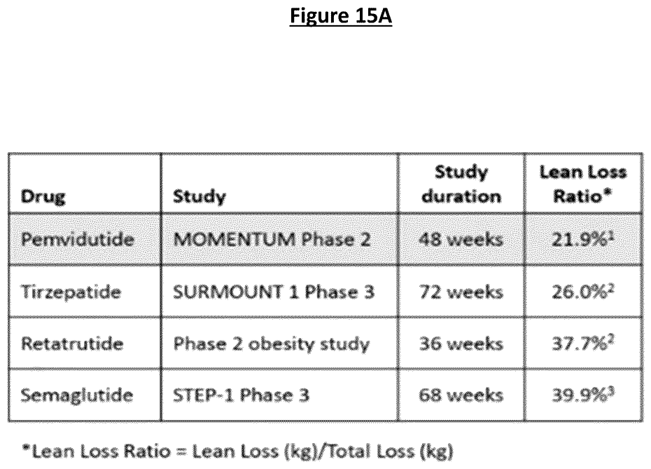

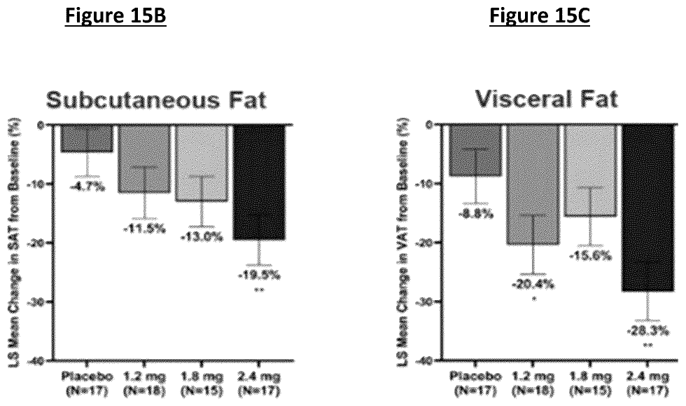

As further detailed in Example 3, in addition to inducing substantial weight loss, pemvidutide treatment resulted in improved lipid profiles, with significant reductions in triglycerides and total cholesterol, and meaningful reductions in serum LDL. The glucagon agonist activity of the present peptide products may be providing the pronounced effect on serum lipids in due to the well-established effects of glucagon on hepatic lipid metabolism as it decreases hepatic lipogenesis, upregulates β-oxidation of fatty acids, increases LDL receptor (LDL-R) expression, and enhances mitochondrial oxidative function. The lipid reductions observed here are greater than those seen in other obesity trials involving incretin-based, mono- and dual-agonists that do not contain glucagon. The combination of GLP-1R/GCGR signaling also yield significant reductions in visceral fat. See FIG. 15.

The disclosure further provides uses of the dual agonist peptide products described herein to prevent and/or treat conditions associated with GLP1R and/or GCGR, such as but not limited to insulin resistance, diabetes, obesity, metabolic syndrome and cardiovascular diseases, and conditions associated therewith, such as NASH and PCOS. In some embodiments, the dual agonist peptide products can be used to treat hyperglycemia, insulin resistance, hyperinsulinemia, prediabetes, diabetes (including types 1 and 2, gestational and juvenile diabetes), diabetic complications, diabetic neuropathy, diabetic nephropathy, diabetic retinopathy, hyperlipidemia, hypercholesterolemia, hypertriglyceridemia, elevated blood levels of free fatty acids, obesity, metabolic syndrome, syndrome X, cardiovascular diseases (including coronary artery disease), atherosclerosis, acute cardiovascular syndrome, ischemia (including myocardial ischemia and cerebral ischemia/stroke), ischemia-reperfusion injury (including myocardial and cerebral IRI), infarction (including myocardial and cerebral infarction), angina, heart failure (e.g., congestive heart failure), peripheral vascular disease, thrombosis (e.g., deep vein thrombosis), embolism (e.g., pulmonary embolism), systemic inflammation (e.g., one characterized by elevated C-reactive protein blood level), and hypertension. The dual agonist peptide products can achieve their therapeutic effects through various mechanisms, including stimulation of blood glucose-dependent insulin secretion, increase in insulin sensitivity, stimulation of fat burning and reduction of body weight. The dual agonist peptide products can also promote, e.g., pancreatic beta-cell protection, cardioprotection and wound healing.

The peptide products described herein can be used to treat other conditions associated with insulin resistance or/and obesity and/or administered concomitantly with drugs used to treat the same. Other conditions associated with insulin resistance or/and obesity include without limitation arthritis (e.g., osteoarthritis), low back pain, breathing disorders (e.g., asthma, obesity hypoventilation syndrome [Pickwickian syndrome] and obstructive sleep apnea), dermatological disorders (e.g., diabetic ulcers, acanthosis nigricans, cellulitis, hirsutism, intertrigo and lymphedema), gastroenterological disorders (e.g., cholelithiasis [gallstone], gastroesophageal reflux disease [GERD] and gastroparesis), gout, hypercortisolism (e.g., Cushing's syndrome), kidney disorders (e.g., chronic kidney disease), liver disorders (e.g., fatty liver disease [FLD] including alcoholic and non-alcoholic FLD), neurological disorders (e.g., carpal tunnel syndrome, dementias [e.g., Alzheimer's disease and vascular dementia], meralgia paresthetica, migraines and multiple sclerosis), urological disorders (e.g., erectile dysfunction, hypogonadism and urinary incontinence), polycystic ovary syndrome, infertility, menstrual disorders, mood disorders (e.g., depression), and cancers (e.g., cancers of the endometrium, esophagus, colorectum, gallbladder, kidney, liver [e.g., hepatocellular carcinoma], pancreas and skin [e.g., melanoma], and leukemia). In certain embodiments, a dual agonist peptide product described herein is used to treat polycystic ovary syndrome (PCOS). In other embodiments, a peptide product is used to treat chronic kidney disease (CKD), also known as chronic kidney/renal failure (CKF/CRF). The most common causes of CKD are diabetes and long-term, uncontrolled hypertension. In further embodiments, a dual agonist peptide product described herein is used to treat fatty liver disease (FLD). In some embodiments, the FLD is non-alcoholic fatty liver disease (NAFLD), also understood to include metabolic fatty liver disease (MFLD). In certain embodiments, the NAFLD is non-alcoholic steatohepatitis (NASH). FLD, also known as hepatic steatosis, is characterized by excessive fat accumulation in the liver. FLD includes alcoholic fatty liver disease (AFLD) and NAFLD. Chronic alcoholism causes fatty liver due to production of toxic metabolites such as aldehydes during metabolism of alcohol in the liver. NAFLD is described below. FLD is associated with diabetes, obesity and metabolic syndrome. Fatty liver can develop into cirrhosis or a liver cancer (e.g., hepatocellular carcinoma [HCC]). Less than about 10% of people with cirrhotic AFLD develop HCC, but up to about 45% of people with NASH without cirrhosis may develop HCC. HCC is the most common type of primary liver cancer in adults and occurs in the setting of chronic liver inflammation. NAFLD is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., dyslipidemia and impaired glucose control). A liver can remain fatty without disturbing liver function, but a fatty liver can progress to become NASH, a condition in which steatosis is accompanied by inflammation, hepatocyte ballooning and cell injury with or without fibrosis of the liver. Fibrosis is the strongest predictor of mortality from NASH. NAFLD can be characterized by steatosis alone; steatosis with lobular or portal inflammation but without ballooning; steatosis with ballooning but without inflammation; or steatosis with inflammation and ballooning. NASH is the most extreme form of NAFLD. NASH is a progressive disease, with about 20% of patients developing cirrhosis of the liver and about 10% dying from a liver disease, such as cirrhosis or a liver cancer (e.g., HCC). NAFLD is the most common liver disorder in developed countries, and NASH is projected to supplant hepatitis C as the major cause of liver transplant in the U.S. by 2020. About 12-25% of people in the U.S. have NAFLD, with NASH affecting about 2-5% of people in the U.S. NAFLD, including NASH, is associated with insulin resistance, obesity and metabolic syndrome. For instance, insulin resistance contributes to progression of fatty liver to hepatic inflammation and fibrosis and thus NASH. Furthermore, obesity drives and exacerbates NASH, and weight loss can alleviate NASH. Therefore, the peptide products described herein, including GLP-1 receptor (GLP1R) agonists, glucagon receptor (GCGR) agonists and dual GLP1R/GCGR agonists, can be used to treat NAFLD, including NASH. In some embodiments, the dual agonist peptide products used to treat a condition associated with insulin resistance or/and obesity disclosed herein, such as fatty liver disease including NAFLD and NASH, is pemvidutide, and/or derivatives thereof, and pharmaceutically acceptable salts thereof.

In some embodiments, the present dual agonist peptide(s) can be used to control blood glucose with reduction of one or more adverse events (i.e., an unexpected event that negatively impacts patient and/or animal welfare). Exemplary, non-limiting adverse events can include nausea, vomiting, diarrhea, abdominal pain and/or constipation, Adverse events may also include any known to those of ordinary skill in the art, such as those listed in industry resources and/or otherwise known to those of ordinary skill in the art (see, e.g., Medical Dictionary for Regulatory Activities (MedDRA) (Pharm, Med. Transl. Med. 2018) and/or Clark, M. J, Biomed. Inf., 54, April 2015, pp. 167-173). Such adverse events can be determined in humans using standard techniques as are typically used in clinical trials (e.g., doctor visit, surveys/questionnaires). As compared to the frequency and/or severity of such an adverse event that occurs upon administration of an agonist with unbalanced affinity for GLP-1R and GCGR (e.g., semaglutide) to a subject, the dual agonist peptides of this disclosure (e.g., any of SEQ ID NO. 1, or derivatives thereof) can decrease such frequency and/or severity thereof by, e.g., 5%, 10%, 20%, 40%, 50%, 60%, 70%, 80%, 90% of higher (up to 100%). In some embodiments, the dual agonist peptides of this disclosure (e.g., pemvidutide) do not cause any adverse events.

A present dual agonist peptide product can be administered by any suitable route for treatment of a condition disclosed herein. Potential routes of administration of a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository)). In some embodiments, a peptide product is administered parenterally, such as subcutaneously, intravenously or intramuscularly. In other embodiments, a peptide product is administered by oral inhalation or nasal inhalation or insufflation. The therapeutically effective amount and the frequency of administration of, and the length of treatment with, a peptide product to treat a condition disclosed herein may depend on various factors, including the nature and severity of the condition, the potency of the compound, the route of administration, the age, body weight, general health, gender and diet of the subject, and the response of the subject to the treatment, and can be determined by the treating physician. In some embodiments, a peptide product is administered parenterally (e.g., subcutaneously (sc), intravenously (iv) or intramuscularly (im)) in a dose from about 0.01 mg to about 0.1, 1, 5 or 10 mg, or about 0.1-1 mg or 1-10 mg, over a period of about one week for treatment of a condition disclosed herein (e.g., one associated with insulin resistance or/and obesity, such as NASH or NAFLD). In further embodiments, a peptide product is administered parenterally (e.g., sc, iv or im) in a dose of about 0.1-0.5 mg, 0.5-1 mg, 1-5 mg or 5-10 mg over a period of about one week. In certain embodiments, a peptide product is administered parenterally (e.g., subcutaneously (SC), intravenous (IV) or intramuscular (IM)) in a dose of about 0.1-1 mg, or about 0.1-0.5 mg or 0.5-1 mg, over a period of about one week. One of skill in the art understands that an effective dose in a mouse, or other pre-clinical animal model, may be scaled for a human. In that way, through allometric scaling (also referred to as biological scaling) a dose in a larger animal may be extrapolated from a dose in a mouse to obtain an equivalent dose based on body weight or body surface area of the animal.

The pemvidutide formulation can be taken substantially with food (e.g., with a meal or within about 1 hour or 30 minutes before or after a meal) or substantially without food (e.g., at least about 1 or 2 hours before or after a meal). The length of treatment of a medical condition with a dual agonist peptide product can be based on, e.g., the nature and severity of the condition and the response of the subject to the treatment and can be determined by the treating physician. In some embodiments, the pemvidutide formulation is administered chronically to treat a condition disclosed herein, such as at least about 2 months, 3 months, 6 months, 1 year, 1.5 years, 2 years, 3 years, 5 years, 10 years or longer. The pemvidutide formulation can also be taken pro re nata (as needed) until clinical manifestations of the condition disappear or clinical targets are achieved, such as blood glucose level, blood pressure, blood levels of lipids, body weight or body mass index, waist-to-hip ratio or percent body fat, or any combination thereof. If clinical manifestations of the condition re-appear or the clinical targets are not maintained, administration of the dual agonist peptide product can resume. The disclosure provides a method of treating a medical condition described herein, comprising administering to a subject in need of treatment a therapeutically effective amount of the pemvidutide formulation described herein or a pharmaceutically acceptable salt thereof, or a pharmaceutical composition comprising the same. The disclosure further provides pemvidutide described herein or a pharmaceutically acceptable salt thereof, or a composition comprising the same, for use as a medicament. In addition, the disclosure provides for the use of pemvidutide described herein or a pharmaceutically acceptable salt thereof in the preparation of a medicament. The medicament containing pemvidutide can be used to treat any medical condition described herein. The pemvidutide can optionally be used in combination with one or more additional therapeutic agents.

Pemvidutide (e.g., as a formulation) can be administered as the sole active agent, or optionally be used in combination with one or more other dual agonist peptide products, and/or additional therapeutic agents to induce weight loss and/or treat any disorder disclosed herein, such as insulin resistance, diabetes, obesity, metabolic syndrome or a CV disease (e.g., heart failure with preserved ejection fraction (HFpEF)), or any condition associated therewith, such as high cholesterol, triglycerides, LDL and/or VLDL particle concentration and/or diameter, and/or other related parameters (e.g., serum phosphatidylethanolamines, phosphatidylcholines, lysophosphatidylethanolamines, sphingolipids and/or lysophosphatidylcholines). In some embodiments, the one or more additional therapeutic agents are selected from antidiabetic agents, anti-obesity agents (including lipid-lowering agents and pro-satiety agents), anti-atherosclerotic agents, anti-inflammatory agents, antioxidants, antifibrotic agents, anti-hypertensive agents, and combinations thereof. Antidiabetic agents include without limitation: AMP-activated protein kinase (AMPK) agonists, including biguanides (e g., buformin and metformin); peroxisome proliferator-activated receptor gamma (PPAR-□) agonists, including thiazolidinediones (e.g., balaglitazone, ciglitazone, darglitazone, englitazone, lobeglitazone, netoglitazone, pioglitazone, rivoglitazone, rosiglitazone and troglitazone), MSDC-0602K and saroglitazar (dual PPAR-□□□□Dagonist); glucagon-like peptide-1 (GLP-1) receptor agonists, including exendin-4, albiglutide, dulaglutide, exenatide, liraglutide, lixisenatide, semaglutide, taspoglutide, CNT0736, CNT03649, HM11260C (LAPS-Exendin), NN9926 (OG9S7GT), TT401 and ZYOGl; dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, teneligliptin, trelagliptin and vildagliptin; sodium-glucose transport protein 2 (SGLT2) inhibitors, including canagliflozin (also inhibits SGLT1), dapagliflozin, empagliflozin, ertugliflozin, ipragliflozin, luseogliflozin, remogliflozin etabonate, sotagliflozin (also inhibits SGLT1) and tofogliflozin; blockers of ATP-dependent K⁺ (KA_{TP}) channels on pancreatic beta cells, including rneglitinides (e.g., mitiglinide, nateglinide and repagiinide) and sulfonylureas {including first generation (e.g., acetohexamide, carbutamide, chlorpropamide, giycyclamide [tolhexamide], metahexamide, tolazamide and tolbutamide) and second generation (e.g., glibenclamide, glyburide, glibornuride, gliclazide, glimepiride, glipizide, gliquidone, glisoxepide and glyclopyramide); insulin and analogs thereof, including fast-acting insulin (e.g., insulin aspari insulin glulisine and insulin lispro), intermediate-acting insulin (e.g., NPH insulin), and long-acting insulin (e.g., insulin degludec, insulin detemir and insulin glargine); and/or, analogs, derivatives and salts thereof. In certain embodiments, the antidiabetic agent is or includes a biguanide (e.g., metformin), a thiazolidinedione (e.g., pioglitazone or rosiglitazone) or a SGLT2 inhibitor (e.g., empagliflozin or tofogliflozin), or any combination thereof. Anti-obesity agents include, but are not limited to: appetite suppressants (anorectics), including amphetamine, dexamphetamine, amfepramone, clobenzorex, mazindol, phentermine (with or without topiramate) and lorcaserin; pro-satiety agents, including ciliary neurotrophic factor (e.g., axokine) and longer-acting analogs of amylin, calcitonin, cholecystokinin (CCK), GLP-1, leptin, oxyntomodulin, pancreatic polypeptide (PP), peptide YY (PYY) and neuropeptide Y (NPY); lipase inhibitors, including caulerpenyne, cetilistat, ebelactone A and B, esterastin, lipstatin, orlistat, percyquinin, panclicin A-E, valilactone and vibralactone; antihyperlipidemic agents; and analogs, derivatives and salts thereof. Antihyperlipidemic agents include without limitation: HMG-CoA reductase inhibitors, including statins {e.g., atorvastatin, cerivastatin, fluvastatin, mevastatin, monacolins (e.g., monacolin K (lovastatin), pitavastatin, pravastatin, rosuvastatin and simvastatin} and flavanones (e.g., naringenin); squalene synthase inhibitors, including lapaquistat, zaragozic acid and RPR-107393; acetyl-CoA carboxylase (ACC) inhibitors, including anthocyanins, avenaciolides, chloroacetylated biotin, cyclodim, diclofop, haloxyfop, soraphens (e.g., soraphen Aia), 5-(tetradecyloxy)-2-furancarboxylic acid (TOFA), CP-640186, GS-0976, NDI-010976; 7-(4-propyloxy-phenylethynyl)-3,3-dimethyl-3,4dihydro-2H-benzo[b][1,4]dioxepine; N-ethyl-N′-(3-{[4-(3,3-dimethyl-1-oxo-2-oxa-7-azaspiro[4.5]dec-7-yl)piperidin-1-yl]-carbonyl}-1-benzothien-2-yl)urea; 5-(3-acetamidobut-1-ynyl)-2-(4-propyloxyphenoxy)thiazole; and 1-(3-{[4-(3,3-dimethyl-1-oxo-2-oxa-7-azaspiro[4.5]dec-7-yl)piperidin-1-yl]-carbonyl}-5-(pyridin-2-yl)-2-thienyl)-3-ethylurea; PPAR-D agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid); PPAR-D agonists, including elafibranor (dual PPAR-□□□□Dagonist), GFT505 (dual PPAR-□□□□□Dagonist), GW0742, GW501516 (dual PPAR-b/δ agonist), sodelglitazar (GW677954), MBX-8025, and isoflavones (e.g., daidzein and genistein); PPAR-□ agonists, including thiazolidinediones {supra), saroglitazar (dual PPAR-□□□□agonist), 4-oxo-2-thioxothiazolines (e.g., rhodanine), berberine, honokiol, perfluorononanoic acid, cyclopentenone prostaglandins (e.g., cyclopentenone 15-deoxy-A-prostaglandin J₂ [15d-PGJ₂]), and isoflavones (e.g., daidzein and genistein); liver X receptor (LXR) agonists, including endogenous ligands (e.g., oxysterols such as 22(i?)-hydroxycholesterol, 24(A)-hydroxy cholesterol, 27-hydroxycholesterol and cholestenoic acid) and synthetic agonists (e.g., acetyl-podocarpic dimer, hypocholamide, A(X-di methyl-3 b-hydroxy-cholenamide [DMHCA], GW3965 and T0901317); retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346); inhibitors of acyl-CoA cholesterol acyltransferase (ACAT, aka sterol G-acyl transferase [SOAT], including ACAT1 [SOAT1] and ACAT2 [SOAT2]), including avasimibe, pactimibe, pellitorine, terpendole C and flavanones (e.g., naringenin); inhibitors of stearoyl-CoA desaturase-1 (SCD-1, aka stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl)thiazol-2-yl]benzamide and 4-ethylamino-3-(2-hydroxyethoxy)-N-[5-(3-trifluoromethylbenzyl)thiazol-2-yl]benzamide; 1′-{6-[5-(pyridin-3-ylmethyl)-1,3,4-oxadiazol-2-yl]pyridazin-3-yl}-5-(trifluoromethyl)-3,4-dihydrospiro[chromene-2,4′-piperidine]; 5-fluoro-1′-{6-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H,3H-spiro[1,5-benzoxazepine-2,4′-piperidin]-1′-yl]-N-(2-hydroxy-2-pyridin-3-ylethyl)pyridazine-3-carboxamide; 6-[4-(2-methylbenzoyl)piperidin-1-yl]pyridazine-3-carboxylic acid (2-hydroxy-2-pyridin-3-ylethyl)amide; 4-(2-chlorophenoxy)-N-[3-(methyl carbamoyl)phenyl]piperidine-1-carboxamide; the cis-9,trans-11 isomer and the trans-10,cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs; cholesterylester transfer protein (CETP) inhibitors, including anacetrapib, dalcetrapib, evacetrapib, torcetrapib and AMG 899 (TA-8995); inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP-targeting microRNAs (e.g., miRNA-30c), and MTTP-targeting siRNAs; GLP-1 receptor agonists; fibroblast growth factor 21 (FGF21) and analogs and derivatives thereof, including BMS-986036 (pegylated FGF21); inhibitors of pro-protein eonvertase subtilisin/kexin type 9 (PCSK9) activity or expression, including berberine (reduces PC8K9 level), annexin A2 (inhibits PCSK9 activity), anti-PCSK9 antibodies (e.g., alirocumab, bococizumab, evolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g, inclisiran [ALN-PCS] and ALN-PCSO2); apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CH1, 5A-CH2, 5A-H1, 18 A, 37 pA A-P-18A], ELK, ELK-1A, ELK-1F, ELK-1K1A1E, ELK-1L1K, ELK-1W, ELK-2A, ELK-2A2K2E, ELK-2E2K, ELK-2F, ELK-3 E3EK, ELK-3E3K3A, ELK-3E3LK, ELK-PA, ELK-P2A, ELKA, ELKA-CH2, ATI-5261, CS-6253, ETC-642, FAMP, FREL and KRES and apoE mimetics (e.g., Ac-hE18A-NH₂, AEM-28, Ac-[R]hEl 8 A-NH₂, AEM-28-14, EpK, hEp, mR18L, COG-112, COG-133 and COG-1410); omega-3 fatty acids, including docosahexaenoic acid (DHA), docosapentaenoic acid (DPA), eicosapentaenoic acid (EPA), a-linolenic acid (ALA), fish oils (which contain, e.g., DHA and EPA), and esters (e.g., glyceryl and ethyl esters) thereof; and analogs, derivatives and salts thereof. In certain embodiments, the anti-obesity agent is or includes a lipase inhibitor (e.g., orlistat) or/and an antihyperlipidemic agent (e.g., a statin such as atorvastatin, or/and a fibrate such as fenofibrate). Antihypertensive agents include without limitation: antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (ATII1) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone); diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothi azide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine; calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyri dines (e.g., diltiazem and verapamil); □₂-adrenoreceptor agonists, including clonidine, guanabenz, guanfacine, methyldopa and moxonidine; □1-adrenoreceptor antagonists (alpha blockers), including doxazosin, indoramin, nicergoline, phenoxybenzamine, phentolamine, prazosin, terazosin and tolazoline; □-adrenoreceptor (□1 or/and □₂) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol; mixed alpha/beta blockers, including bucindolol, carvedilol and labetalol; endothelin receptor antagonists, including selective ETA receptor antagonists (e.g., ambrisentan, atrasentan, edonentan, sitaxentan, zibotentan and BQ-123) and dual ET_{A}/ET_{B} antagonists (e.g., bosentan, macitentan and tezosentan); other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin Ei (alprostadil) and analogs thereof (e.g., limaprost amd misoprostol), prostacyclin and analogs thereof (e.g., ataprost, beraprost [e.g., esuberaprost], 5,6,7-trinor-4,8-inter-w-phenylene-9-fluoro-PG₂, carbacyclin, isocarbacyclin, clinprost, ciprostene, eptaloprost, cicaprost, iloprost, pimilprost, SM-10906 (des-methyl pimilprost), naxaprostene, taprostene, treprostinil, CS-570, OP-2507 and TY-11223), non prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, and protein kinase C (PKC) inhibitors (e.g., BIM-1, BIM-2, BIM-3, BIM-8, cherythrine, cicletanine, gossypol, miyabenol C, myricitrin, ruboxistaurin and verbascoside; minerals, including magnesium and magnesium sulfate; and analogs, derivatives and salts thereof. In certain embodiments, the antihypertensive agent is or includes a thiazide or thiazide like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof. In some embodiments, a peptide product described herein is used in combination with one or more additional therapeutic agents to prevent and/or treat CV disease-related conditions, such as high cholesterol, triglycerides, LDL and/or VLDL particle concentration and/or diameter, and/or other related parameters (e.g., serum phosphatidylethanolamines, phosphatidylcholines, lysophosphatidylethanolamines, sphingolipids and/or lysophosphatidylcholines). In some embodiments, the one or more additional therapeutic agents are selected from antidiabetic agents, anti-obesity agents, anti-inflammatory agents, antifibrotic agents, antioxidants, anti-hypertensive agents, and combinations thereof. Therapeutic agents that can be used to treat NAFLD (e.g., NASH) include without limitation: PPAR agonists, including PPAR-δ agonists (e.g., MBX-8025, elafibranor [dual PPAR-α/δ agonist] and GW501516 [dual PPAR-b/δ agonist]) and PPAR-γ agonists (e.g., thiazolidinediones such as pioglitazone, and saroglitazar [dual PPAR-α/γ agonist])-PPAR-δ and -γ agonism increases insulin sensitivity, PPAR-α agonism reduces liver steatosis and PPAR-δ agonism inhibits activation of macrophages and Kupffer cells; farnesoid X receptor (FXR) agonists, such as obeticholic acid and nonsteroidal FXR agonists like GS-9674 reduce liver gluconeogenesis, lipogenesis, steatosis and fibrosis; fibroblast growth factor 19 (FGF19) and analogs and derivatives thereof, such as NGM-282-FGF19 analogs reduce liver gluconeogenesis and steatosis; fibroblast growth factor 21 (FGF21) and analogs and derivatives thereof, such as BMS-986036 (pegylated FGF21)-FGF21 analogs reduce liver steatosis, cell injury and fibrosis; HMG-CoA reductase inhibitors, including statins (e.g., rosuvastatin)—statins reduce steatohepatitis and fibrosis; ACC inhibitors, such as NDI-010976 (liver-targeted) and GS-0976-ACC inhibitors reduce de novo lipogenesis and liver steatosis; SCD-1 inhibitors, such as aramchol—SCD-1 inhibitors reduce liver steatosis and increase insulin sensitivity; SGLT2 inhibitors, such as canagliflozin, ipragliflozin and luseogliflozin-SGLT2 inhibitors reduce body weight, liver ALT level and fibrosis; antagonists of CCR2 or/and CCR5, such as cenicriviroc-antagonists of CCR2 (binds to CCL2 [MCP1]) and CCR5 (binds to CCL5 [RANTES]) inhibit activation and migration of inflammatory cells (e.g., macrophages) to the liver and reduce liver fibrosis; apoptosis inhibitors, including apoptosis signal-regulating kinase 1 (ASK1) inhibitors (e.g., selonsertib) and caspase inhibitors (e.g., emricasan [pan-caspase inhibitor])-apoptosis inhibitors reduce liver steatosis and fibrosis; lysyl oxidase-like 2 (LOXL2) inhibitors, such as simtuzumab-LOXL2 is a key matrix enzyme in collagen formation and is highly expressed in the liver; galectin-3 inhibitors, such as GR-MD-02 and TD139-galectin-3 is critical for development of liver fibrosis; antioxidants, including vitamin E (e.g., a-tocopherol) and scavengers of reactive oxygen species (ROS) and free radicals (e.g., cysteamine, glutathione, melatonin and pentoxifylline [also anti-inflammatory via inhibition of TNF-a and phosphodiesterases])-vitamin E reduces liver steatosis, hepatocyte ballooning and lobular inflammation; and, analogs, derivatives and salts thereof. In some embodiments, a peptide product described herein is used in conjunction with a PPAR agonist (e.g., a PPAR-δ agonist such as elafibranor or/and a PPAR-γ agonist such as pioglitazone), a HMG-CoA reductase inhibitor (e.g., a statin such as rosuvastatin), an FXR agonist (e.g., obeticholic acid) or an antioxidant (e.g., vitamin E), or any combination thereof, to treat NAFLD (e.g., NASH). In certain embodiments, the one or more additional therapeutic agents for treatment of NAFLD (e.g., NASH) are or include vitamin E or/and pioglitazone. Other combinations may also be used as would be understood by those of ordinary skill in the art.

Pharmacokinetic (“PK”) parameters can be estimated using Phoenix@WinNonlin® version 8.1 or higher (Certara USA, Inc., Princeton, New Jersey). A non-compartmental approach consistent with the extravascular route of administration can be used for parameter estimation. The individual plasma concentration-time data can be used for pharmacokinetic calculations. In addition to parameter estimates for individual animals, descriptive statistics (e.g. mean, standard deviation, coefficient of variation, median, min, max) can be determined, as appropriate. Concentration values that are below the limit of quantitation can be treated as zero for determination of descriptive statistics and pharmacokinetic analysis. Embedded concentration values that are below the limit of quantitation can be excluded from pharmacokinetic analysis. All parameters can be generated from individual dual agonist peptide (or derivatives and/or metabolites thereof) concentrations in plasma from test article-treated groups on the day of dosing (Day 1). Parameters can be estimated using nominal dose levels, unless out of specification dose formulation analysis results are obtained, in which case actual dose levels can be used. Parameters can be estimated using nominal sampling times; if bioanalytical sample collection deviations are documented, actual sampling times can be used at the affected time points. Bioanalytical data can be used as received for the pharmacokinetic analysis and can be presented in tables and figures in the units provided. Pharmacokinetic parameters can be calculated and presented in the units provided by the analytical laboratory (the order of magnitude can be adjusted appropriately for presentation in the report, e.g. h*ng/mL converted to h*μg/mL). Descriptive statistics (e.g., mean, standard deviation, coefficient of variation, median, min, max) and pharmacokinetic parameters can be determined to three significant figures, as appropriate. Additional data handling items can be documented as needed. PK parameters to be determined, as data permit, can include but are not limited to the following: Cₘₐₓ: Maximum observed concentration; DN Cₘₐₓ: dose normalized maximum concentration, calculated as Cₘₐₓ/dose; Tₘₐₓ: time of maximum observed concentration; AUC₀₋ₜ: area under the curve from time 0 to the time of the last measurable concentration, calculated using the linear trapezoidal rule; AUC₀₋₉₆: area under the curve from time 0 to hour 96, calculated using the linear trapezoidal rule; DN AUC₀₋₉₆: dose normalized AUC₀₋₉₆, calculated as AUC₀₋₉₆/dose; AUC_{0-inf}: area under the curve from time 0 to infinity (Day 1 only), calculated as AUC_{0-inf}=AUC₀₋ₜ+Cₜ/λz, where Cₜ is the last observed quantifiable concentration and λ_{z} is the elimination rate constant; t_{1/2}: elimination half-life, calculated as ln(2)/λ_{z}. Additional parameters and comparisons (e.g., sex ratios, dose proportionality ratios, etc.) can also be determined, as would be understood by those of ordinary skill in the art.

In some embodiments, this disclosure provides pharmaceutical dosage formulation(s) comprising pemvidutide wherein the peptide product is modified with a hydrophobic surfactant; the dosage is configured to induce weight loss, with reduction of one or more adverse events wherein the subject is at risk for and/or has a CV disease-related conditions (and/or CV disease) and may also be overweight, obese, and/or suffer from type 2 diabetes, wherein the adverse events being selected from nausea, vomiting, diarrhea, abdominal pain and constipation, upon administration to a mammal. In certain embodiments, the formulation comprises 1.2, 1.8, or 2.4 mg of pemvidutide as a weekly therapeutic dose (the 2.4 mg dose being preferably preceded by titration up to 2.4 mg for four weeks (preferably 0.6 mg during week 1, 1.2 mg per week during week 2, 1.8 mg per week during week 3, and 1.8 mg per week during week 4), preferably weekly for 48 weeks.

“Reducing,” or “reduction of” adverse effects or events refers to a reduction in the degree, duration, and/or frequency of adverse effects experienced by a subject and incidence in a group of subjects following administration of an agonist with about balanced affinity to GLP1R and GCGR. Such reduction encompasses the prevention of some adverse effects that a subject would otherwise experience in response to an agonist with unbalanced affinity to GLP1R and GCGR. Such reduction also encompasses the elimination of adverse effects previously experienced by a subject following administration of an agonist with unbalanced affinity to GLP1R and GCGR. In some embodiments, “reducing,” or “reduction of” adverse effects encompass a reduction of gastrointestinal side effects wherein the adverse events are reduced to zero or undetectable levels. In other embodiments, adverse effect is reduced to level equivalent to untreated subjects but not completely eliminated. Moreover, administration of analogs with unbalanced affinity toward GLP-1R or GCGR to a mammal may lead to the need for an excessively high dose to maximally activate the receptor with weaker sensitivity toward the ligand, thus leading to a potential for exceeding the biologically effective dose level for the other ligand and causing dose-related, undesired side effects.

In preferred embodiments, this disclosure provides methods of reducing body weight in a human being, wherein the method comprises administering pemvidutide once weekly in an amount from at least 1.2 mg up to 2.4 mg (preferably 1.2 mg, 1.8 mg, or 2.4 mg with titration up to 2.4 mg) to the human being in need thereof; and, wherein the human being is overweight, obese, and/or may also suffer from type 2 diabetes (and, in some preferred embodiments, exhibits symptoms of or markers associated with cardiovascular (CV) disease). In some preferred embodiments of such methods, the pemvidutide is administered once weekly in an amount of 2.4 mg (following titration to the 2.4 mg dose) and the body weight of the human being decreases by at least about 15% (e.g., preferably at least about 15.6%) (i.e., “robust mean weight loss”). In preferred embodiments, the patients exhibit substantial and clinically meaningful reductions in one or more of total cholesterol, low density lipoproteins, triglycerides, and/or blood pressure. In preferred embodiments, within a population of patients to whom the 2.4 mg weekly dose has been administered, at least about 30% or more of the patients lose about least about 20% or more body weight at week 48. In preferred embodiments, continued administration of the 2.4 weekly dose, or in some embodiments a lesser dose, continues to induce weight loss in a patient until a desired weight is achieved. In some embodiments, a maintenance dose (e.g., in preferred embodiments, a 1.2, 1.8 or 2.4 mg weekly dose) may be administered to the patient, either after the initial 48-week administration is completed, or after the desired weight is achieved. In preferred embodiments, the patient(s) experience no gastrointestinal (GI) or cardiac (e.g., arrythmia, increased heart rate) adverse events. In preferred embodiments, where a patient(s) experiences GI adverse events, such event is considered by the patient and/or an attending medical professional as mild to moderate in severity. In preferred embodiments, the patient also exhibits stable glucose homeostasis.

In some preferred embodiments of this method the administration of pemvidutide (preferably 2.4 mg per week for 48 weeks, including a 4-week titration) induces a significant reduction in atherogenic lipids and/or lipoprotein number as compared to baseline. In some preferred embodiments of this method, the atherogenic lipoprotein number concentrations are reduced by at least −0.2 log₂ fold change as compared to baseline following 43 and/or 84 days of administration of pemvidutide; the total serum triglyceride concentration of the subject is reduced by at least −0.2 log₂ fold change relative to placebo following 43 and/or 84 days of administration of pemvidutide; wherein the serum lipids are selected from glycerolipids, sterols, glycerophospholipids, and sphingolipids, optionally wherein the reductions are at least −0.2 log₂ fold change. In some preferred embodiments of this method a reduction in serum phosphatidylethanolamine, phosphatidylcholine, lysophosphatidylethanolamine, sphingolipids and/or lysophosphatidylcholine is induced.

In certain embodiments of this method, the body weight of the human being to whom pemvidutide is administered in a therapeutically effective dose from about 1.2 mg up to 2.4 mg once weekly dosing for 48 weeks (including a 4-week titration for the 2.4 mg dose) is reduced by at least 10% or at least 11%, or at least 12%, or at least 13%, or at least 14%, or at least 15% from baseline at week 48. In certain embodiments of this method, the human being has type 2 diabetes; and/or the human being has a body mass index (BMI kg/m²) of at least 25, at least about 28, or at least about 30.

In certain embodiments provided herein are methods for reducing serum lipids comprising administering to a human subject in need thereof a once weekly therapeutically effective dose of at least 1.2 mg and up to 2.4 mg of pemvidutide for at least 48 weeks. In embodiments, serum triglycerides are reduced after 48 weeks of once weekly administration of a therapeutically effective dose of pemvidutide by at least 20%, at least 25% or at least 30% from baseline values. In embodiments, total serum cholesterol is reduced after 48 weeks of once weekly administration of a therapeutically effective dose of pemvidutide by at least 5%, at least 10%, or at least 15%. In embodiments, serum LDL is reduced after 48 weeks of once weekly administration of a therapeutically effective dose of pemvidutide by at least 10%, at least 15% or at least 20%. In embodiments, serum VLDL is reduced after 48 weeks of once weekly administration of a therapeutically effective dose of pemvidutide by at least 10%, at least 15%, at least 20% or at least 25%. In embodiments, the pemvidutide is administered as a liquid pharmaceutical formulation comprising SEQ ID NO: 1 and about 0.20% (w/w) polysorbate 20, about 0.348% (w/w) arginine, and about 4.260% (w/w) mannitol in sterile water (pH 7.7±0.1).

In some preferred embodiments, the pemvidutide is administered by parenteral injection. In some preferred embodiments, the pemvidutide is administered by subcutaneous injection. In some preferred embodiments, the human being is overweight, is obese, and/or has a body mass index (BMI kg/m²) of at least 27. In some preferred embodiments, the human being has a body mass index (BMI kg/m²) of 30 or greater. In some preferred embodiments, the human being has level of a parameter related to a CV disease-related condition of 10% or greater before treatment (e.g., at baseline). In some preferred embodiments, the absolute reduction in a CV disease-related condition is any of about 8%, 10%, 12% or preferably about 15% after 48 weeks treatment, as compared to measured values at baseline. In some preferred embodiments, the relative reduction in a CV disease-related condition to baseline is greater than about any of 40%, 50% or 60% after 48 weeks treatment. In some preferred embodiments, the pemvidutide is administered from a liquid comprising at least about 1.8 mg/ml and up to 2.4 mg/mL of pemvidutide. In some embodiments, the pemvidutide is administered from a pharmaceutical dosage form as an aqueous formulation comprising one or more of polysorbate 20, Arginine, or Mannitol.

In preferred embodiments, this disclosure relates to methods for administering pemvidutide to a patient in a weekly dose of 1.2 mg, 1.8 mg, or 2.4 mg (in preferred embodiments including a 4-week titration for the 2.4 mg dose) for 48 weeks, preferably 2.4 mg per week, wherein the body weight of the human being is reduced by at least at about 15% (preferably at least 15.6%) from baseline at week 48. In preferred embodiments, administration of 2.4 mg pemvidutide is administered to a population of human beings for at least 48 weeks (including the 4-week titration) wherein, at week 48, the body weight of at least about 30% of the population is reduced by greater than or equal to about 20% from baseline. In preferred embodiments, the patient is overweight, obese and/or has a body mass index (BMI kg/m²) of greater than or equal to 25; and/or optionally has Type II diabetes. In preferred embodiments, the weight loss is accompanied by substantial and clinically meaningful reductions in serum lipids, cholesterol, triglycerides, LDL and/or VLDL particle concentration and/or diameter, phosphatidylethanolamines, phosphatidylcholines, lysophosphatidylethanolamines, sphingolipids and/or lysophosphatidylcholines in serum of the human being, and/or blood pressure, as those parameters can be determined by one of ordinary skill in the art using routine techniques. In some preferred embodiments, the administration of pemvidutide at 2.4 mg per week (including the 4-week titration) reduces cardiovascular (CV) risk for the patient, and/or the population of patients. In some embodiments, the pemvidutide is administered by subcutaneous (parenteral) injection. In some preferred embodiments, the pemvidutide is administered from a liquid formulation comprising at least about 1.8 mg/ml pemvidutide. In some preferred embodiments, the pemvidutide is administered as a liquid pharmaceutical formulation comprising SEQ ID NO: 1 and about 0.20% (w/w) polysorbate 20, about 0.348% (w/w) arginine, and about 4.260% (w/w) mannitol in sterile water (pH 7.7±0.1).

### Specific Embodiments

Specific Embodiment 1: A method for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1, and wherein the human being in need thereof has a co-morbidity selected from one or more of cardiovascular disease, hypertension, dyslipidemia, dysglycemia, and obstructive sleep apnea.

Specific Embodiment 2: The method of embodiment 1, wherein the treatment further reduces serum lipids.

Specific Embodiment 3: A method for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1, and wherein the treatment further reduces serum lipids.

Specific Embodiment 4: The method of embodiment 3, wherein the human being in need thereof has a co-morbidity selected from one or more of cardiovascular disease, hypertension, dyslipidemia, dysglycemia, and obstructive sleep apnea.

Specific Embodiment 5: The method of any preceding embodiment, wherein the peptide product is at least 85%, at least 90%, at least 95%, at least 96%, at least 97%, at least 98% or at least 99% identical to a peptide of SEQ ID NO: 1.

Specific Embodiment 6: The method of any preceding embodiment, wherein the peptide product is 100% identical to a peptide of SEQ ID NO: 1.

Specific Embodiment 7: The method of any preceding embodiment, wherein the pharmaceutical dosage formulation is administered subcutaneously or by parenteral injection.

Specific Embodiment 8: The method of any preceding embodiment, wherein the pharmaceutical dosage is an aqueous formulation comprising one or more of polysorbate 20, arginine, or mannitol.

Specific Embodiment 9: The method of any preceding embodiment, wherein the pharmaceutical dosage formulation comprises about 0.025-0.5% (w/w) polysorbate 20, about 0.2-0.5% (w/w) arginine, and about 3-6% (w/w) mannitol in water (pH 7.7±0.1).

Specific Embodiment 10: The method of any preceding embodiment, wherein the pharmaceutical dosage formulation comprises about 0.2% (w/w) polysorbate 20, about 0.348% (w/w) arginine, about 4.260% (w/w) mannitol in water (pH 7.7±0.1).

Specific Embodiment 11: The method of any preceding embodiment, wherein the human being is obese or overweight.

Specific Embodiment 12: The method of any preceding embodiment, wherein the human being in need thereof has a body mass index (BMI kg/m²) of at least 25.

Specific Embodiment 13: The method of any preceding embodiment, wherein the human being in need thereof has a body mass index (BMI kg/m²) of at least 30.

Specific Embodiment 14: The method of any preceding embodiment, wherein the human being in need thereof has a body mass index (BMI kg/m²) of at least 35.

Specific Embodiment 15: The method of any preceding embodiment, wherein the human being in need thereof has a body mass index (BMI kg/m²) of at least 40.

Specific Embodiment 16: The method of any preceding embodiment, wherein the treatment maintains weight loss.

Specific Embodiment 17: The method of any preceding embodiment, wherein the treatment reduces relative body weight by at least about 10%, by at least about 15% or by at least about 20%.

Specific Embodiment 18: The method of any preceding embodiment, wherein the treatment reduces lean body mass by less than about 25%.

Specific Embodiment 19: The method of any preceding embodiment, wherein the human being in need thereof has an HbA1C value of about 5.7% to 6.4%.

Specific Embodiment 20: The method of embodiment 1-14, wherein the human being in need thereof has an HbA1C value of about 4.0% to 5.6%.

Specific Embodiment 21: The method of any preceding embodiment, wherein the pharmaceutical dosage formulation comprises about 1.2 mg of the peptide product.

Specific Embodiment 22: The method of any preceding embodiment, wherein the pharmaceutical dosage formulation comprises about 1.8 mg of the peptide product.

Specific Embodiment 23: The method of any preceding embodiment, wherein the pharmaceutical dosage formulation comprises about 2.4 mg of the peptide product.

Specific Embodiment 24: The method of any preceding embodiment, wherein the pharmaceutical dosage formulation comprises (2.4 mg/ml; 0.24% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Specific Embodiment 25: The method of any preceding embodiment, wherein the pharmaceutical dosage formulation comprises (1.8 mg/ml; 0.18% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Specific Embodiment 26: The method of any preceding embodiment, wherein the pharmaceutical dosage formulation comprises (1.2 mg/ml; 0.12% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Specific Embodiment 27: The method of any preceding embodiment, wherein the treatment reduces one or more serum lipids selected from serum triglycerides, total serum cholesterol, serum LDL, or serum VLDL.

Specific Embodiment 28: The method of any preceding embodiment, wherein the treatment reduces serum triglycerides by at least about 30% after 48 weeks.

Specific Embodiment 29: The method of any preceding embodiment, wherein the treatment reduces total serum cholesterol by at least about 10% after 48 weeks.

Specific Embodiment 30: The method of any preceding embodiment, wherein the treatment reduces total serum cholesterol by at least about 15% after 48 weeks.

Specific Embodiment 31: The method of any preceding embodiment, wherein the treatment reduces serum LDL by at least about 10% after 48 weeks.

Specific Embodiment 32: The method of any preceding embodiment, wherein the treatment reduces serum VLDL by at least about 25% after 48 weeks.

Specific Embodiment 33: The method of any preceding embodiment, wherein the treatment reduces serum triglycerides by at least about 30% after 48 weeks in human being in need thereof with >150 mg/dL triglycerides at baseline.

Specific Embodiment 34: The method of any preceding embodiment, wherein the treatment reduces serum triglycerides by at least about 50% after 48 weeks in human being in need thereof with >150 mg/dL triglycerides at baseline.

Specific Embodiment 35: The method of any preceding embodiment, wherein the treatment reduces serum cholesterol by at least about 15% after 48 weeks in human being in need thereof with >200 mg/dL cholesterol at baseline.

Specific Embodiment 36: The method of any preceding embodiment, wherein the treatment reduces serum cholesterol by at least about 20% after 48 weeks in human being in need thereof with >200 mg/dL cholesterol at baseline.

Specific Embodiment 37: The method of any preceding embodiment, wherein the treatment reduces serum LDL by at least about 15% after 48 weeks in human being in need thereof with >130 mg/dL LDL at baseline.

Specific Embodiment 38: The method of any preceding embodiment, wherein the treatment reduces serum LDL by at least about 20% after 48 weeks in human being in need thereof with >130 mg/dL LDL at baseline.

Specific Embodiment 39: The method of any preceding embodiment, wherein a steady state therapeutic dose is achieved after a dose escalating phase having a duration of from two to four weeks.

Specific Embodiment 40: A method for treatment of reducing serum lipids in a human being in need thereof, comprising administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1.

Specific Embodiment 41: The method of embodiment 40, wherein the human being in need thereof has a co-morbidity selected from one or more of obesity, cardiovascular disease, hypertension, dyslipidemia, dysglycemia, type 2 diabetes and obstructive sleep apnea.

Specific Embodiment 42: The method of embodiment 40 or 41, wherein the peptide product is at least 85%, at least 90%, at least 95%, at least 96%, at least 97%, at least 98% or at least 99% identical to a peptide of SEQ ID NO: 1.

Specific Embodiment 43: The method of embodiment 40 or 41, wherein the peptide product is 100% identical to a peptide of SEQ ID NO: 1.

Specific Embodiment 44: The method of embodiments 40-43, wherein the pharmaceutical dosage formulation is administered subcutaneously or by parenteral injection.

Specific Embodiment 45: The method of embodiments 40-44, wherein the pharmaceutical dosage is an aqueous formulation comprising one or more of polysorbate 20, arginine, or mannitol.

Specific Embodiment 46: The method of any embodiments 40-45, wherein the pharmaceutical dosage formulation comprises about 0.025-0.5% (w/w) polysorbate 20, about 0.2-0.5% (w/w) arginine, and about 3-6% (w/w) mannitol in water (pH 7.7±0.1).

Specific Embodiment 47: The method of any embodiments 40-46, wherein the pharmaceutical dosage formulation comprises about 0.2% (w/w) polysorbate 20, about 0.348% (w/w) arginine, about 4.260% (w/w) mannitol in water (pH 7.7±0.1).

Specific Embodiment 48: The method of any embodiments 40-47, wherein the human being is obese or overweight.

Specific Embodiment 49: The method of any embodiments 40-48, wherein the human being in need thereof has a body mass index (BMI kg/m²) of at least 25.

Specific Embodiment 50: The method of any embodiments 40-49, wherein the human being in need thereof has a body mass index (BMI kg/m²) of at least 30.

Specific Embodiment 51: The method of any embodiments 40-50, wherein the human being in need thereof has a body mass index (BMI kg/m²) of at least 35.

Specific Embodiment 52: The method of any embodiments 40-51, wherein the human being in need thereof has a body mass index (BMI kg/m²) of at least 40.

Specific Embodiment 53: The method of any embodiments 40-52, wherein the treatment maintains weight loss.

Specific Embodiment 54: The method of any embodiments 40-53, wherein the treatment reduces relative body weight by about at least 10%, by at least 15% or by at least 20%.

Specific Embodiment 55: The method of any embodiments 40-54, wherein the treatment reduces lean body mass by less than about 25%.

Specific Embodiment 56: The method of any embodiments 40-55, wherein the human being in need thereof has an HbA1C value of about 5.7% to 6.4%.

Specific Embodiment 57: The method of embodiments 40-56, wherein the human being in need thereof has an HbA1C value of about 4.0% to 5.6%.

Specific Embodiment 58: The method of embodiments 40-57, wherein the pharmaceutical dosage formulation comprises about 1.2 mg of the peptide product.

Specific Embodiment 59: The method of embodiments 40-58, wherein the pharmaceutical dosage formulation comprises about 1.8 mg of the peptide product.

Specific Embodiment 60: The method of embodiments 40-59, wherein the pharmaceutical dosage formulation comprises about 2.4 mg of the peptide product.

Specific Embodiment 61: The method of embodiments 40-60, wherein the pharmaceutical dosage formulation comprises (2.4 mg/ml; 0.24% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Specific Embodiment 62: The method of embodiments 40-61, wherein the pharmaceutical dosage formulation comprises (1.8 mg/ml; 0.18% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Specific Embodiment 63: The method of embodiments 40-62, wherein the pharmaceutical dosage formulation comprises (1.2 mg/ml; 0.12% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

Specific Embodiment 64: The method of embodiments 40-63, wherein the treatment reduces one or more of serum triglycerides, total serum cholesterol, serum LDL, or serum VLDL.

Specific Embodiment 65: The method of embodiments 40-64, wherein the treatment reduces serum triglycerides by at least about 20% after 48 weeks.

Specific Embodiment 66: The method of embodiments 40-65, wherein the treatment reduces serum triglycerides by at least about 30% after 48 weeks.

Specific Embodiment 67: The method of embodiments 40-66, wherein the treatment reduces total serum cholesterol by at least about 10% after 48 weeks.

Specific Embodiment 68: The method of embodiments 40-67, wherein the treatment reduces total serum cholesterol by at least about 15% after 48 weeks.

Specific Embodiment 69: The method of embodiments 40-68, wherein the treatment reduces serum LDL by at least about 10% after 48 weeks.

Specific Embodiment 70: The method of embodiments 40-69, wherein the treatment reduces serum LDL by at least about 20% after 48 weeks.

Specific Embodiment 71: The method of embodiments 40-70, wherein the treatment reduces serum VLDL by at least about 25% after 48 weeks.

Specific Embodiment 72: The method of embodiments 40-71, wherein the treatment reduces serum triglycerides by at least about 30% after 48 weeks in human being in need thereof with >150 mg/dL triglycerides at baseline.

Specific Embodiment 73: The method of embodiments 40-72, wherein the treatment reduces serum triglycerides by at least about 50% after 48 weeks in human being in need thereof with >150 mg/dL triglycerides at baseline.

Specific Embodiment 74: The method of embodiments 40-73, wherein the treatment reduces serum cholesterol by at least about 15% after 48 weeks in human being in need thereof with >200 mg/dL cholesterol at baseline.

Specific Embodiment 75: The method of embodiments 40-74, wherein the treatment reduces serum cholesterol by at least about 20% after 48 weeks in human being in need thereof with >200 mg/dL cholesterol at baseline.

Specific Embodiment 76: The method of embodiments 40-75, wherein the treatment reduces serum LDL by at least about 15% after 48 weeks in human being in need thereof with >130 mg/dL LDL at baseline.

Specific Embodiment 77: The method of embodiments 40-76, wherein the treatment reduces serum LDL by at least about 20% after 48 weeks in human being in need thereof with >130 mg/dL LDL at baseline.

Specific Embodiment 78: The method of embodiments 40-77, wherein a steady state therapeutic dose is achieved after a dose escalating phase having a duration of from two to four weeks.

Other aspects of this disclosure are also contemplated as will be understood by those of ordinary skill in the art.

Unless defined otherwise or clearly indicated otherwise by their use herein, all technical and scientific terms used herein have the same meaning as commonly understood by those of ordinary skill in the art to which this application belongs. As used in the specification and the appended claims, the word “a” or “an” means one or more. As used herein, the word “another” means a second or more. The acronym “aka” means also known as. The term “exemplary” as used herein means “serving as an example, instance or illustration”. Any embodiment or feature characterized herein as “exemplary” is not necessarily to be construed as preferred or advantageous over other embodiments or features. In some embodiments, the term “about” or “approximately” means within +10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Optional or optionally means that the subsequently described event or circumstance can or cannot occur, and that the description includes instances where the event or circumstance occurs and instances where it does not. All publications, patents, and patent applications mentioned in this specification are herein incorporated by reference in their entireties to the same extent as if each individual publication, patent, or patent application was specifically and individually indicated to be incorporated by reference.

Certain embodiments are further described in the following examples. These embodiments are provided as examples only and are not intended to limit the scope of the claims in any way.

### EXAMPLES

### Example 1. Peptide Synthesis

The peptide and peptide products disclosed herein can be made in accordance with well-known procedures and by processes known and disclosed in the art. For example, SEQ ID NO: 1 can be prepared in accordance with synthetic protocols provided in U.S. Pat. No. 12,171,806. The contents of Example 1 of U.S. Pat. No. 12,171,806 are herein incorporated by reference.

### Example 2: 48-Week Phase 2 Obesity Trial of Pemvidutide (Momentum Study: NCT05295875) Study Design

### Introduction

Obesity is a chronic disease that has risen to epidemic proportions worldwide, with an estimated 3.3 billion people projected to be affected by 2035. Obesity is associated with risk factors for other conditions, including dyslipidemia, hypertension, type 2 diabetes mellitus (T2DM), and metabolic dysfunction-associated steatohepatitis (MASH). A 5% reduction in bodyweight has been associated with improvements in cardiovascular risk factors, T2DM, steatotic liver disease, and other obesity related co-morbidities, whereas loss of 10 to 15% bodyweight is associated with reductions in cardiovascular mortality, T2DM regression, and histologic improvements of MASH activity.

Bariatric surgery is historically the most efficacious weight-reduction treatment as meaningful levels of weight loss have been difficult to achieve pharmacologically. However, the advent of incretin-based therapies have demonstrated the potential to achieve significantly greater weight loss compared to earlier medical interventions. Glucagon-like peptide-1 receptor (GLP-1R) agonists (e.g., semaglutide) have been used to treat T2DM for over a decade, and more recently, two agents (e.g. semaglutide and tirzepatide) that activate the GLP-1R were approved by the U.S. Food and Drug Administration (FDA) for chronic weight management.

Applicant herein provides a different strategy to achieve additional levels of weight loss—to combine GLP-1R agonists with co-agonists having complementary mechanisms. GLP-1R agonists achieve weight loss by anorectic effects, suppressing food consumption. G-coupled glucagon receptor (GCGR) agonists are recognized to increase energy expenditure and shift metabolism toward increased utilization of lipids as an energy supply, potentially mimicking the effects of diet and exercise. Accordingly, provided herein are methods using peptide products of this disclosure, (e.g., GLP-1R/GCGR dual agonists) providing mechanisms for further reducing body weight beyond GLP-1 monotherapy.

The study was a Phase 2, multi-center, randomized, double-blind, placebo-controlled, parallel-group dose-finding trial designed to evaluate the effects of ALT-801 (pemvidutide) on body weight, tolerability, and safety. The study was conducted at 30 research sites in the USA. The trial was conducted in accordance with the principles of the Declaration of Helsinki and the International Conference on Harmonization Good Clinical Practice. The trial protocol and amendments were approved by the relevant institutional review boards and/or ethics committees at each study site. All participants provided written informed consent prior to participation in the study.

### Inclusion Criteria

Participants were eligible for the study if they had provided written informed consent prior to entry. They had to be between 18 and 75 years old and have a body mass index (BMI) of at least 30 kg/m² or a BMI of at least 27 kg/m² with at least one obesity-related comorbidity, such as cardiovascular disease, hypertension, dyslipidemia, pre-diabetes, or obstructive sleep apnea. Pre-diabetes was defined as an HbA1c level between 5.7% and 6.4%, fasting glucose between 100 and 125 mg/dL (5.5-6.9 mmol/L), or a glucose tolerance test result between 140 and 199 mg/dL (7.7-11.0 mmol/L). Participants were required to have had at least one unsuccessful weight loss attempt as determined by the investigator and had to agree to comply with the study's lifestyle interventions and treatments for the full duration of the study. Female participants of childbearing potential were eligible if they had a negative pregnancy test, were not breastfeeding, and agreed to use effective contraception during the study and for one month after. Acceptable methods included hormonal contraceptives or intrauterine devices. Women who were surgically sterile or postmenopausal were also eligible. Male participants with female partners of childbearing potential had to be surgically sterile or have partners meeting the study's criteria. They also agreed not to donate sperm during the study and for three months after the last dose. Participants with same-sex partners or practicing abstinence were eligible.

### Exclusion Criteria

Participants were excluded if they had experienced significant weight fluctuations, specifically a weight gain or loss of more than 5% in the three months prior to randomization or more than 10% in the six months prior to screening. Those with any form of diabetes, including Type 1, Type 2, or mature-onset diabetes of the young (MODY), were not eligible, though a history of gestational diabetes was allowed if glucose levels were currently normal. Obesity-related conditions, including those induced by genetic causes or endocrine disorders such as Cushing Syndrome or untreated hypothyroidism, led to exclusion, as did any history of bariatric surgery or other surgical weight-loss interventions. Gastrointestinal and liver conditions, including pancreatitis, significant GI disorders, surgeries altering absorption or transit, inflammatory bowel disease, or cirrhosis, also resulted in exclusion. Additionally, those with a history of pheochromocytoma, insulinoma, or glucagonoma were ineligible. Candidates with a recent history of neoplastic disease (within five years) were excluded unless the condition had been adequately treated, such as non-melanomatous skin carcinoma or benign cervical neoplasia under proper surveillance. A personal or family history of medullary thyroid carcinoma or multiple endocrine neoplasia also disqualified participation. Mental health conditions, including untreated depressive disorders (PHQ-9 score ≥15), suicidal ideation, prior suicide attempts, or severe psychiatric disorders such as schizophrenia or bipolar disorder, resulted in exclusion. Participants with cardiovascular conditions, including poorly controlled hypertension, conduction abnormalities (QT prolongation), recent myocardial infarction, stroke, or congestive heart failure, were not eligible. Those with clinically significant laboratory abnormalities were also excluded, including impaired renal function (eGFR<60 mL/min/1.73 m²), elevated liver enzymes (ALT or AST>2×ULN), alkaline phosphatase (ALP) values >2×ULN, total bilirubin >1.3 mg/dL (except Gilbert Syndrome), international normalized ratio (INR)>1.3, fasting serum triglycerides >500 mg/dL, hemoglobin <11.0 g/dL, platelet count <125,000/μL, or any other abnormality deemed to have exceeded normal safety limits as determined by the Investigator. Infectious conditions such as HIV, active hepatitis B or C, and recent COVID-19 infection were disqualifying factors. A history of substance abuse, a positive toxicology screening for certain drugs, or recreational intravenous drug use also led to exclusion. The use of medications affecting glucose metabolism, weight loss, or herbal remedies within specific time frames resulted in disqualification unless deemed appropriate by the investigator. Participants who had received an investigational product within 90 days or had severe allergic reactions, hypersensitivity to GLP-1 analogues, or any medical, psychiatric, or social condition that may have interfered with adherence to the study were not eligible. Lastly, individuals who, in the investigator's opinion, were unlikely or unwilling to comply with the study protocol were not included.

### Randomization and Stratification

Eligible subjects were randomized by interactive web response system (IWRS) in a 1:1:1:1 ratio to one of four treatment arms: Group (1) ALT-801 1.2 mg subcutaneously (SC) once weekly for 48 weeks, Group (2) ALT-801 1.8 mg SC once weekly for 48 weeks, Group (3) ALT-801 0.6 mg SC for 1 week, followed by 1.2 mg SC for 1 week, then 1.8 mg SC once weekly for 2 weeks, and 2.4 mg SC once weekly for 44 weeks, or Group (4) placebo SC once weekly for 48 weeks. Randomization was stratified based on sex and baseline body mass index (BMI<35 kg/m2 vs. ≥35 kg/m2), targeting a minimum of 25% of randomized subjects being male. Participants were required to escalate to or maintain the target dose without suspending the titration scheme or reducing the dose for intolerability. However, treatment could be held for up to 2 weeks if intolerability was experienced.

### Study Medications

Study medication was provided as pre-filled syringes (PFS) for administration and all syringes were matched for volume and appearance. The investigator, participant, and study staff remained blinded to treatment assignments throughout the study. Pemvidutide Injection in PFS is a sterile solution in prefilled syringes (1 mL/syringe). Each prefilled syringe contains 1 mL of drug product solution at the specified strengths and is intended for SC administration. The quantitative information for the excipients (mannitol, arginine, and polysorbate 20) in the PFS formulations are the same as the Pemvidutide Injection in vials. Pemvidutide Injection in PFS is presented in 3 different strengths (1.2, 1.8, and 2.4 mg/mL) for SC administration. The different strengths are accomplished by formulating different concentrations of pemvidutide with the same syringe fill volume of 1 mL. The quantitative compositions of the 3 strengths are presented in Table 1.

TABLE 1
Quantitative Composition - Pemvidutide Injection in PFS

| | | 1.2 mg/mL | | 1.8 mg/mL | | 2.4 mg/mL | |
|---|---|---|---|---|---|---|---|
| Component | | Quantity | | Quantity | | Quantity | |
| and Quality | | per Unit | | per Unit | | per Unit | |
| Standard | Function | (mg/mL) | % (w/v) | (mg/mL) | % (w/v) | (mg/mL) | % (w/v) |
| Pemvidutide | API | 1.2 | 0.12 | 1.8 | 0.18 | 2.4 | 0.24 |
| Mannitol | Tonicity | 42.6 | 4.26 | 42.6 | 4.26 | 42.6 | 4.26 |
| USP, Ph. Eur. | agent | | | | | | |
| Arginine | Buffer | 3.48 | 0.348 | 3.48 | 0.348 | 3.48 | 0.348 |
| USP, Ph. Eur. | | | | | | | |
| Polysorbate 20 | Surfactant | 2 | 0.2 | 2 | 0.2 | 2 | 0.2 |
| NF, Ph. Eur. | | | | | | | |
| HCl 1N | For pH | For pH | — | For pH | — | For pH | — |
| NF, Ph. Eur. | adjustment | adjustment | | adjustment | | adjustment | |
| | | to 7.75 | | to 7.75 | | to 7.75 | |
| Sterile Water | Solvent | Q.S to 1.0 | 95.072 | Q.S to 1.0 | 95.012 | Q.S to 1.0 | 94.952 |
| USP, Ph. Eur. | | mL | | mL | | mL | |
| Total | — | 1 mL | 100 | 1 mL | 100 | 1 mL | 100 |

### Procedures

Throughout the study, subjects attended regularly scheduled visits, which were conducted via telehealth when feasible. The investigational product (IP) was administered according to protocol guidelines. Subjects received counseling on a reduced-calorie diet of 1200-1500 calories per day for individuals weighing <250 lbs (113.6 kg) and 1500-1800 calories for those ≥250 lbs (113.6 kg). A gradual increase in physical activity, targeting 150 minutes per week, was recommended. Compliance with lifestyle interventions was regularly assessed by the Investigator. Investigators adhered to predefined decision criteria regarding the timing and method of intervention for subjects who developed worsening abnormal liver function tests. Dose interruptions were monitored and discussed with the medical monitor, when necessary, with reasons including illness, inability to attend clinic visits, AEs, and laboratory abnormalities. Subjects with dose interruptions due to safety or tolerability concerns had the option to permanently discontinue treatment, while being encouraged to continue study assessments. Subjects were educated on recognizing and managing hypoglycemia and instructed to use a glucometer to measure random glucose levels if symptoms occurred. If glucose readings fell below 70 mg/dL (3.9 mmol/L), subjects contacted the study Investigator. Treatment for hyperglycemia, defined as fasting glucose ≥180 mg/dL (≥10 mmol/L), was conducted according to predefined protocols. Body composition changes were assessed via magnetic resonance imaging (MRI) in a sub-study. Sparse blood samples were collected to analyze ALT-801 pharmacokinetic and to assess immunogenicity and anti-drug antibody (ADA) titer for efficacy correlation. Subjects who failed screening due to non-allowed prior or concomitant medications were eligible for rescreening at the discretion of the Medical Monitor. Laboratory values outside acceptable ranges could be re-evaluated once during screening. Blood pressure criteria for eligibility could be reassessed with one additional reading within the screening period. An unblinded interim analysis was conducted when approximately 160 subjects completed 24 weeks of treatment or withdrew early. The study's safety was overseen by an independent Data Monitoring Committee (iDMC), responsible for adjudicating major adverse cardiac events (MACE) and AEs related to acute pancreatitis. The iDMC Charter detailed their role and responsibilities. Measures to minimize COVID-19 risks were communicated to participants. Subjects were not prevented from receiving approved or emergency-use authorized COVID-19 vaccines or treatments during trial participation. A ±2-day window for IP administration was allowed if a COVID-19 vaccine was given near a scheduled dose. Height, bodyweight, waist circumference, and vital signs (systolic and diastolic blood pressure and heart rate) were measured at baseline, and with the exception of height, these measurements were repeated every 4 weeks or less throughout the trial. Additional assessments included fasting glucose, HbA1c, and serum lipids

### Endpoints

The primary efficacy endpoint was the relative change from baseline in body weight (%) at 48 weeks. Secondary endpoints included the proportion of subjects achieving ≥5%, ≥10%, ≥15%, and ≥20% weight loss at 48 weeks. Additional secondary efficacy endpoints included changes from baseline in fasting lipids (total cholesterol, HDL, IDL, LDL, VLDL cholesterol, Apo AI, Apo AII, Apo B, lipoprotein (a), and triglycerides), glucose homeostasis and insulin resistance (HbA1c, fasting glucose, fasting insulin, HOMA-IR), systolic and diastolic blood pressure, and use of concomitant antihypertensive medications. Exploratory endpoints included body composition measured by MRI in a sub-study. Metabolic markers and inflammatory markers were also analyzed. Additionally, patient-reported outcomes related to weight-related quality of life and general health status were assessed using IWQOL-Lite, SF-36, and PGI-C. Safety endpoints included adverse events (AEs) such as GI tolerability (nausea, vomiting, diarrhea, constipation), hyperglycemia, hypoglycemia, injection site reactions, and Major Adverse Cardiac Events MACE (nonfatal stroke, nonfatal myocardial infarction, or cardiovascular death). Vital signs, including heart rate, blood pressure, and rate-pressure product, were monitored. Safety labs (liver function tests, glucose, amylase, and lipase), 12-lead ECG, physical examination, and immunogenicity (ADA titers) were also evaluated. Pharmacokinetic (PK) endpoints included ALT-801 population PK analysis and PK-PD modeling.

### Example 3: Results from the 48-Week Phase 2 Obesity Trial of Pemvidutide (Momentum Study: NCT05295875)

The trial enrolled 391 subjects 18-75 year old human males and females (at least approximately 25% male) with obesity (BMI≥30 kg/m²) or overweight (BMI≥27 kg/m²) with at least one obesity-related co-morbidity (history of cardiovascular disease, hypertension, dyslipidemia, pre-diabetes (e.g., dysglycemia), and/or obstructive sleep apnea) and without diabetes (HbA1c≤6.5% and fasting glucose ≤125 mg/dl), with at least one unsuccessful weight loss attempt as detailed in example 2. See FIGS. 1-2. At baseline, subjects had a mean age of approximately 50 years, mean body mass index (BMI) of approximately 37 kg/m² and mean body weight of approximately 104 kg.

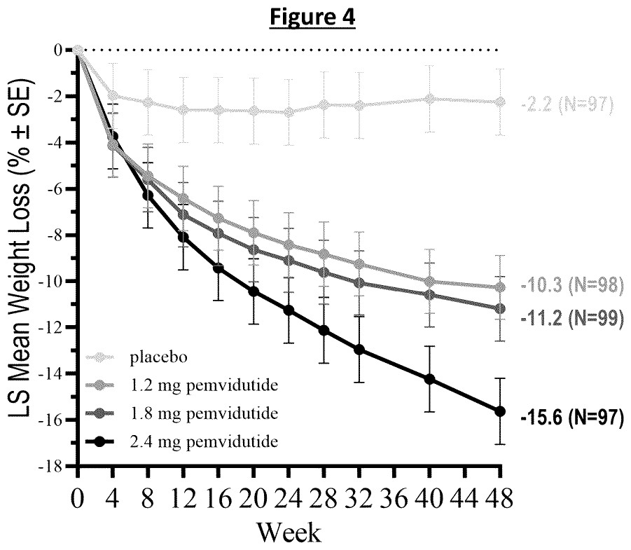

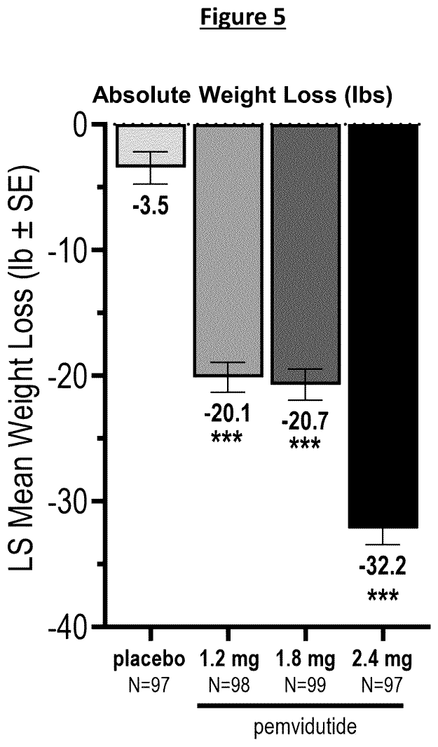

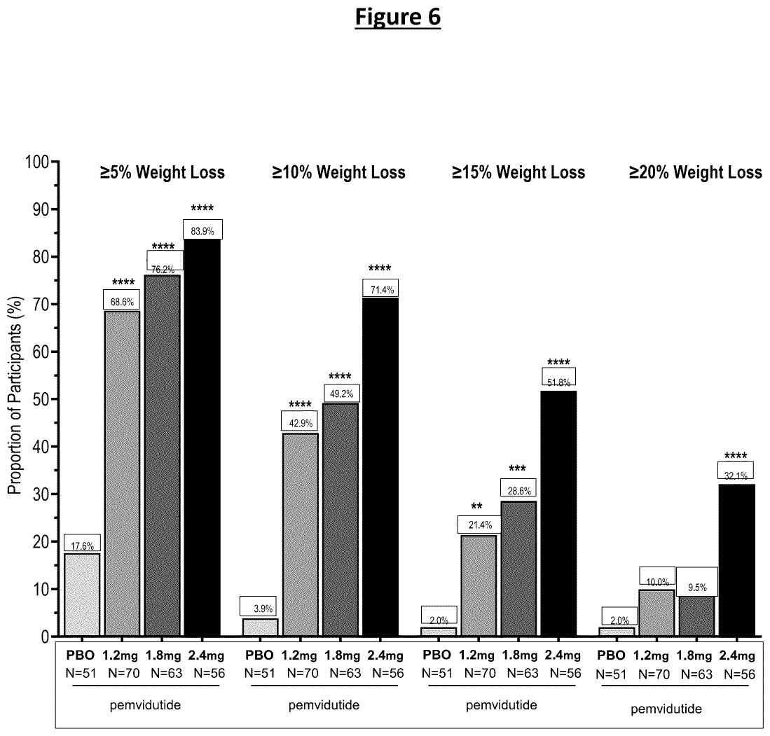

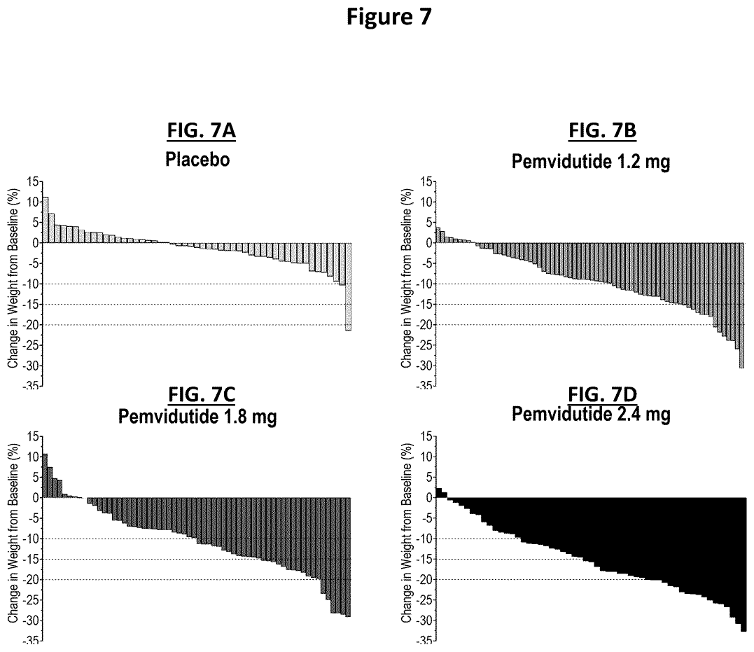

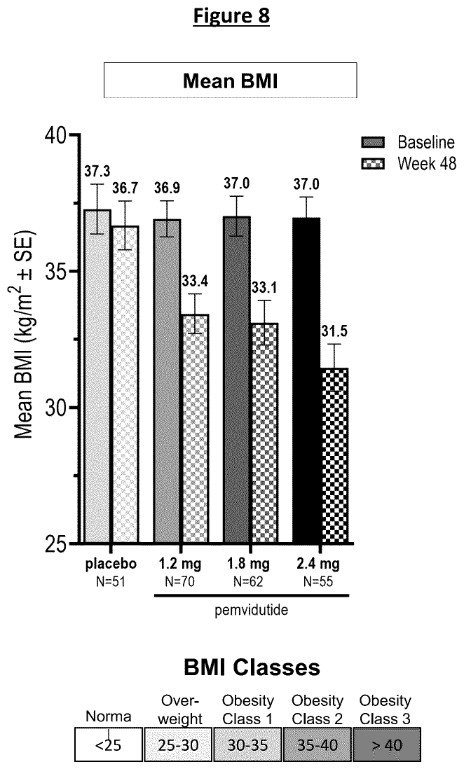

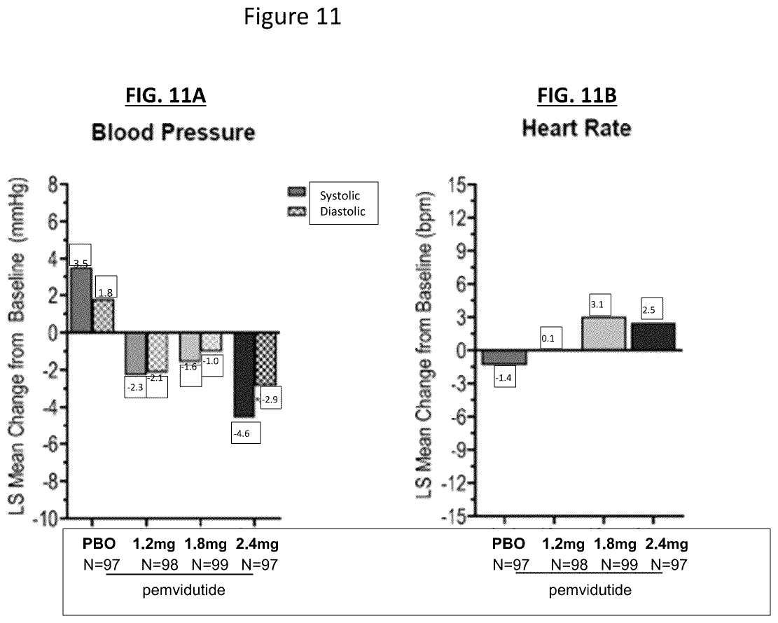

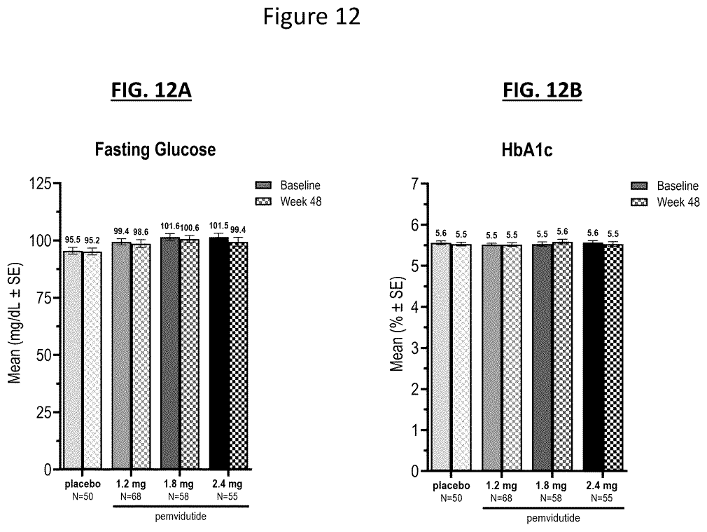

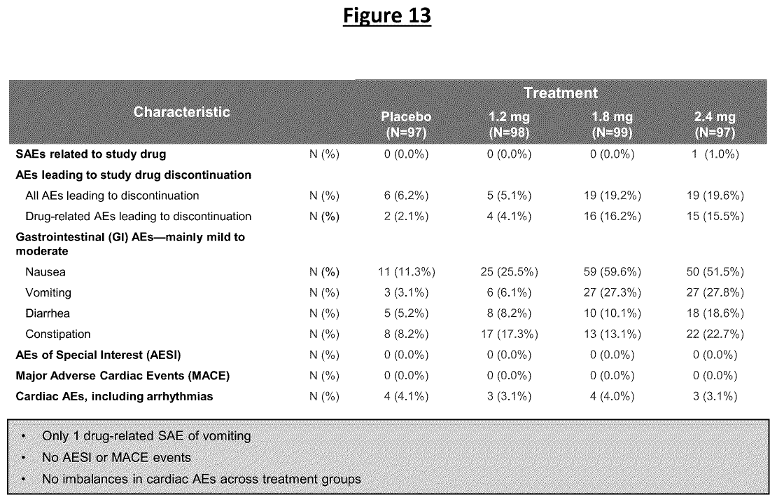

At Week 48, subjects receiving pemvidutide (i.e., SEQ ID NO: 1) achieved relative weight losses of 10.3%, 11.2%, 15.6% and 2.2% at the 1.2 mg, 1.8 mg, and 2.4 mg doses and placebo, respectively, with a near-linear trajectory of continued weight loss observed on the 2.4 mg dose at the end of treatment. See FIGS. 3-4. At Week 48, subjects receiving pemvidutide achieved absolute weight losses of 3.5%, 20.1%, 20.7% and 32.2% at the 1.2 mg, 1.8 mg, and 2.4 mg doses and placebo, respectively. See FIG. 5. Over 50% of subjects achieved at least 15% weight loss and over 30% of subjects achieved at least 20% weight loss on the 2.4 mg dose. See FIGS. 6-7. Significant reductions in BMI were also observed at week 48. See FIG. 8. Pemvidutide administered weekly for 48 weeks resulted in robust reductions in serum lipids (See FIGS. 9-10 (subjects with elevated baseline levels)) and improvements in blood pressure without imbalances in cardiac events, arrhythmias or clinically meaningful increases in heart rate. See FIG. 11. Glucose homeostasis was maintained, with no significant changes in fasting glucose or HbA1c. See FIG. 12. More subjects receiving pemvidutide stayed on study compared to those receiving placebo, with 74.1% of pemvidutide subjects completing the trial compared to 61.9% of placebo subjects. Nausea and vomiting comprised the majority of adverse events (AEs) and were predominantly mild to moderate in severity. See FIG. 13. Only one (1.0%) subject experienced a drug-related serious adverse event (SAE), a case of vomiting at the 2.4 mg dose. Rates of AEs leading to treatment discontinuation were 6.2% in subjects receiving placebo and 5.1%, 19.2%, and 19.6% in subjects receiving 1.2 mg, 1.8 mg and 2.4 mg of pemvidutide, respectively. Study discontinuations related to study drug occurred in 2.1% of placebo subjects and 4.1%, 16.2% and 15.5% in subjects receiving 1.2 mg, 1.8 mg and 2.4 mg of pemvidutide, respectively, with most discontinuations due to AEs in the pemvidutide groups occurring in the first 16 weeks of treatment. No AEs of special interest or major adverse cardiac events (MACE) were observed, and there were low rates of cardiac AEs, including arrhythmias, with no imbalance across pemvidutide or placebo groups. A summary of the efficacy findings are shown in Tables 2-5 and a summary of safety and tolerability findings are shown in Tables 6 and 7 below.

TABLE 2

| Primary Endpoint: | | Placebo | 1.2 mg | 1.8 mg | 2.4 mg |
|---|---|---|---|---|---|
| Body Weight | | (N = 97) | (N = 98) | (N = 99) | (N = 97) |
| ΔBodyweight, | %, LSM | −2.2 | −10.3 | −11.2 | −15.6 |
| all subjects | (SE)¹ | (1.4) | (1.4)*** | (1.4)*** | (1.4)*** |

TABLE 3

| | | Placebo | 1.2 mg | 1.8 mg | 2.4 mg |
|---|---|---|---|---|---|
| Responder Analyses | | (N = 51) | (N = 70) | (N = 63) | (N = 56) |
| % Subject w/≥5% weight loss | %² | 17.6% | 68.6%**** | 76.2%**** | 83.9%**** |
| % Subject w/≥10% weight loss | | 3.9% | 42.9%**** | 49.2%**** | 71.4%**** |
| % Subject w/≥15% weight loss | | 2.0% | 21.4%** | 28.6%*** | 51.8%**** |
| % Subject w/≥20% weight loss | | 2.0% | 10.0% | 9.5% | 32.1%**** |

TABLE 4

| | | Placebo | 1.2 mg | | 1.8 mg | | 2.4 mg | |
|---|---|---|---|---|---|---|---|---|
| Secondary Endpoints | | (N = 50) | (N = 69) | | (N = 58) | | (N = 55) | |
| Δ Total cholesterol | %, LSM | −2.8 (2.0) | −11.6 | (1.7)** | −13.1 | (1.9)** | −15.1 | (2.0)*** |
| Δ LDL cholesterol | (SE)³ | −2.8 (4.1) | −6.2 | (3.5) | −11.2 | (3.8) | −9.9 | (3.9) |
| Δ Triglycerides | | +7.3 (4.6) | −21.7 | (3.9)*** | −22.3 | (4.3)*** | −34.9 | (4.4)*** |

TABLE 5

| | | Placebo | 1.2 mg | 1.8 mg | 2.4 mg |
|---|---|---|---|---|---|
| Blood Pressure and Heart Rate | | (N = 97) | (N = 98) | (N = 99) | (N = 97) |
| Δ Systolic BP | mm Hg, | +3.5 (2.3) | −2.3 (2.2) | −1.6 (2.2) | −4.6 (2.3) |
| Δ Diastolic | LSM (SE)¹ | +1.8 (1.4) | −2.1 (1.3) | 1.0 (1.3) | −2.9 (1.4) |
| Δ Heart Rate | bpm, | −1.4 (1.6) | 0.1 (1.5) | 3.1 (1.5) | 2.5 (1.6) |
| | LSM (SE)¹ | | | | |

¹MMRM (mixed model for repeated measures),
²CMH (Cochran Mantel Haenszel),
³ANCOVA (analysis of covariance)
*p < 0.05;
**p < 0.005;
***p < 0.001;
****p < 0.0001 compared with placebo

TABLE 6

| | | Placebo | | 1.2 mg | | 1.8 mg | | 2.4 mg | |
|---|---|---|---|---|---|---|---|---|---|
| Adverse events (AEs) | | (N = 97) | | (N = 98) | | (N = 99) | | (N = 97) | |
| SAEs related to study drug | N (%) | 0 | (0.0%) | 0 | (0.0%) | 0 | (0.0%) | 1 | (1.0%)⁴ |
| All AEs leading to | N (%) | 6 | (6.2%) | 5 | (5.1%) | 19 | (19.2%) | 19 | (19.6%) |
| discontinuation | | | | | | | | | |
| Drug-related AEs leading | N (%) | 2 | (2.1%) | 4 | (4.1%) | 16 | (16.2%) | 15 | (15.5%) |
| to discontinuation | | | | | | | | | |

Gastrointestinal AEs---mainly mild to moderate

| | | | | | | | | | |
|---|---|---|---|---|---|---|---|---|---|
| Nausea | N (%) | 11 | (11.3%) | 25 | (25.5%) | 59 | (59.6%) | 50 | (51.5%) |
| Vomiting | N (%) | 3 | (3.1%) | 6 | (6.1%) | 27 | (27.3%) | 27 | (27.8%) |
| Diarrhea | N (%) | 5 | (5.2%) | 8 | (8.2%) | 10 | (10.1%) | 18 | (18.6%) |
| Constipation | N (%) | 8 | (8.2%) | 17 | (17.3%) | 13 | (13.1%) | 22 | (22.7%) |
| Major Adverse Cardiac | N (%) | 0 | (0.0%) | 0 | (0.0%) | 0 | (0.0%) | 0 | (0.0%) |
| Events (MACE) | | | | | | | | | |
| Cardiac AEs Including | N (%) | 4 | (4.1%) | 3 | (3.1%) | 4 | (4.0%) | 3 | (3.1%) |
| arrhythmias | | | | | | | | | |

⁴Vomiting

TABLE 7

| | | | | |
|---|---|---|---|---|
| | Placebo | 1.2 mg | 1.8 mg | 2.4 mg |
| Summary of Glycemic Control | (N = 50) | (N = 68) | (N = 58) | (N = 55) |

Fasting glucose

| | | | | | | | | | |
|---|---|---|---|---|---|---|---|---|---|
| Baseline, mg/dL | mean (SE) | 95.5 | (1.5) | 99.4 | (1.4) | 101.6 | (1.4) | 101.5 | (1.6) |
| Week 48, mg/dL | mean (SE) | 95.2 | (1.5) | 98.6 | (1.7) | 100.6 | (1.6) | 99.4 | (2.0) |

HbA1c

| | | | | | | | | | |
|---|---|---|---|---|---|---|---|---|---|
| Baseline, % | mean (SE) | 5.6 | (0.0) | 5.5 | (0.0) | 5.5 | (0.1) | 5.6 | (0.0) |
| Week 48, % | mean (SE) | 5.5 | (0.0) | 5.5 | (0.0) | 5.6 | (0.1) | 5.5 | (0.1) |

In summary, this data shows robust mean weight loss of 15.6% on pemvidutide following administration of 2.4 mg per week for 48 wks, with mean and maximal weight loss of 32.2 and 87.1 lbs, respectively. Over 30% of subjects lost 20% or more body weight following administration of 2.4 mg per week for 48 weeks. Continued weight loss was also observed following administration of 2.4 mg per week for 48 weeks. Substantial and clinically meaningful reductions in total cholesterol, LDL, triglycerides and blood pressure were also observed. With respect to safety and tolerability, GI AEs, common to incretin-based agents were mainly mild to moderate in severity, no imbalance of cardiac AEs (including arrhythmias) were observed, no clinically meaningful increases in heart rate were observed, and glucose homeostasis was maintained.

Provided herein in certain embodiments are methods for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1, and wherein the human being in need thereof has a co-morbidity selected from one or more of cardiovascular disease, hypertension, dyslipidemia, dysglycemia, and obstructive sleep apnea.

Provided herein in certain embodiments are methods for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1, and wherein the treatment further reduces serum lipids.

Provided herein in certain embodiments are method for treatment of reducing serum lipids in a human being in need thereof, comprising administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1.

### Example 4: Body Composition

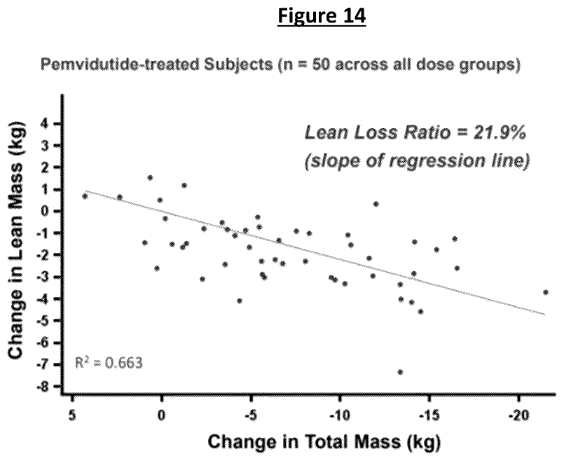

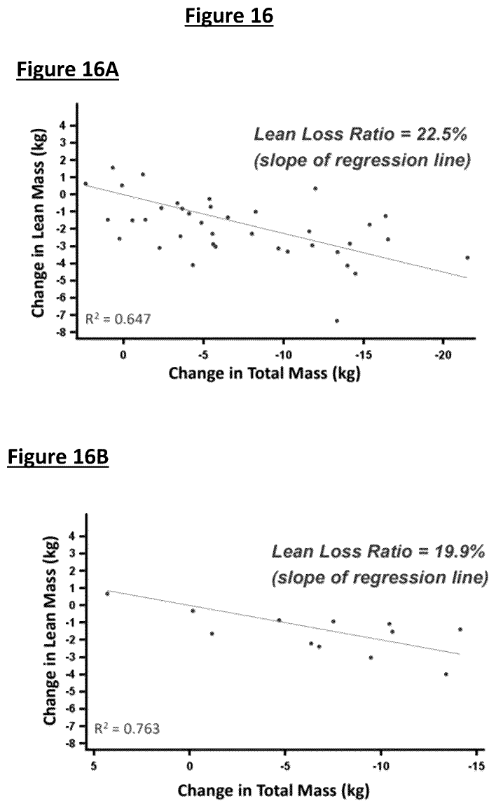

A comprehensive Magnetic Resonance Imaging (MRI)-based body composition analysis was conducted in a subpopulation of 50 subjects from Example 2 both before and after treatment. The results indicate that pemvidutide (SEQ ID NO: 1)-treated subjects exhibited an average lean loss ratio (defined as the change in lean mass divided by the change in total mass) of approximately 21.9%. See FIG. 14. This ratio is lower than values reported for diet and exercise alone, as well as for other weight loss agents such as tirzepatide, semaglutide, and retatrutide. See FIG. 15A. Notably, older subjects maintained similar levels of lean mass preservation. See FIG. 16. The reduced lean mass in older subjects may be beneficial in reducing the risk of falls and fractures, a known concern when lean mass is lost to a significant extent. Preserving lean mass during weight reduction is critical, as excessive lean mass loss has been linked to adverse outcomes, including sarcopenia and bone fractures, especially in women and the elderly. In addition, the body composition analysis revealed that the majority of weight loss (78.1%) was attributable to fat reduction. More specifically, visceral adipose tissue—an adipose tissue depot associated with cardiovascular risk—was reduced by 28.3% at the 2.4 mg pemvidutide dose. See FIG. 15B. Subcutaneous adipose tissue was also reduced by 19.5% at this dose. See FIG. 15C. Overall, the MRI-based body composition analysis confirms the preferential loss of adipose tissue over lean tissue in subjects treated with pemvidutide.

### Example 5: Lipidomics

Lipidomics analyses were performed on plasma samples from subjects from Momentum Study NCT05295875. Analysis was performed on placebo group (N=52), 1.2 mg dose group (n=72), 1.8 mg dose group (n=69) and 2.4 mg dose group (n=61) comparing baseline, week 24 and week 48. Lipidomics (Liposcale) consisted in using ultra-high performance liquid chromatography—mass spectrometry (UHPLC-MS) to detect changes 470 lipids species across 63 lipid classes. Considering the extent of the dataset, Table 8 only presents the results for lipid species regrouped in 63 lipid class from the 2.4 mg dose group in terms of Log 2 fold change (FC) and statistical significance (P) at week 24 and week 48 compared to baseline. Within 24 weeks of treatment, pemvidutide significantly reduced serum lipid levels across all lipid classes, especially glycerolipids (diglycerides and triglycerides), glycerophospholipids (phosphatidylethanolamines, phosphatidylcholines, lysophosphatidylethanolamines, and lysophosphatidylcholines) and sphingolipids (ceramides and sphingomyelins) associated with reduction in cardiovascular (CV) risk as well as reduction in insulin resistance. Obesity has been shown to be associated with increased circulating levels of glycerolipids, including diglycerides (DGs) and triglycerides (TGs). This can represent a potential risk factor for comorbidities, as DGs are considered lipotoxic species related to hepatic insulin resistance, and elevated triglycerides levels are strongly associated with inadequate glycemic control and related to increased risks of diabetes, cardiovascular diseases or MASLD/MASH. Obesity is commonly associated with abnormal levels of circulating free fatty acids that is reduced in the context of pemvidutide treated. Pemvidutide also reduces plasma bile acids (BA) that has been shown to have a positive correlation with body mass index. Pemvidutide induced consistent decreases in most sterols whereas no changes were detected in the placebo arm. Regarding PEs, pemvidutide treatment caused consistent decreases in the great majority of diacyl-,lyso- and ether-linked forms of these glycerophospholipids in comparison to the baselines, both after 24 and 48 weeks of intervention. With regard to the phosphatidylcholines (PCs), placebo treatment only led to minor significant changes, regardless of the PC class. pemvidutide treatment exerted consistent reductions in the whole PC profile. Pemvidutide also caused major decreases in ceramides (Cer), sphingomyelins (SMs), monohexosylceramides (CMHs) and free sphingoid bases (FSB). Similar trends were observed for the 1.2 mg and 1.8 mg doses while limited to no significant changes were observed in the placebo group.

TABLE 8

| | | 2.4 mg W 24 | | 2.4 mg W 48 | |
|---|---|---|---|---|---|
| | | vs. Baseline | | vs. WO | |
| Group | Species | FC | P | FC | P |
| FA | Fatty acids | −0.21 | 8.2E−03 | −0.22 | 6.0E−02 |
| | Saturated fatty acids | −0.11 | 4.1E−02 | −0.12 | 1.5E−01 |
| | Monounsaturated fatty acids | −0.27 | 3.4E−02 | −0.25 | 1.1E−01 |
| | Polyunsaturated fatty acids | −0.23 | 3.7E−03 | −0.25 | 3.2E−02 |
| | Unsaturated fatty acids | −0.23 | 5.9E−03 | −0.25 | 5.6E−02 |
| | Fatty acids omega 3 | −0.37 | 1.9E−04 | −0.38 | 1.7E−03 |
| | Fatty acids omega 6 | −0.09 | 6.9E−02 | −0.15 | 2.6E−01 |
| | Fatty acids omega 9 | −0.13 | 8.0E−02 | −0.18 | 2.6E−01 |
| Ox. FA | Oxidized fatty acids | −0.25 | 2.2E−04 | −0.10 | 5.9E−02 |
| AC | Acylcarnitines | −0.08 | 4.8E−01 | 0.08 | 6.2E−02 |
| GL | Diglycerides | −0.33 | 4.0E−05 | −0.46 | 2.6E−07 |
| | Saturated Diglycerides | −0.52 | 5.6E−05 | −0.43 | 3.9E−05 |
| | Unsaturated Diglycerides | −0.33 | 8.3E−05 | −0.49 | 2.4E−07 |
| | Monounsaturated (1sat) Diglycerides | −0.87 | 9.1E−08 | −0.90 | 9.1E−08 |
| | Polyunsaturated (>2sat) Diglycerides | −0.23 | 3.2E−03 | −0.38 | 3.4E−06 |
| | Triglycerides | −0.36 | 1.4E−06 | −0.66 | 1.2E−06 |
| | Saturated Triglycerides | −0.56 | 9.1E−04 | −0.92 | 1.1E−02 |
| | Unsaturated Triglycerides | −0.37 | 9.3E−07 | −0.64 | 5.2E−07 |
| | Monounsaturated (1sat) Triglycerides | −0.78 | 1.2E−04 | −1.11 | 2.4E−04 |
| | Polyunsaturated (>3sat) Triglycerides | −0.42 | 3.5E−06 | −0.60 | 1.2E−07 |
| BA | Bile Acids | −0.43 | 3.7E−03 | −0.45 | 9.6E−02 |
| | Free bile acids | −0.33 | 1.5E−01 | −0.34 | 4.9E−01 |
| | Taurine-conjugated bile acids | −0.61 | 1.8E−03 | −0.85 | 1.5E−02 |
| | Glycine-conjugated bile acids | −0.33 | 1.7E−02 | −0.21 | 6.0E−02 |
| ST | Cholesteryl Esters | −0.23 | 9.6E−05 | −0.14 | 3.8E−03 |
| | Saturated Cholesteryl Esters | −0.22 | 5.2E−04 | −0.18 | 1.1E−02 |
| | Monounsaturated Cholesteryl Esters | −0.45 | 5.6E−07 | −0.41 | 8.5E−06 |
| | Polyunsaturated Cholesteryl Esters | −0.14 | 4.4E−03 | −0.09 | 3.0E−02 |
| | Steroids | −0.42 | 2.0E−09 | −0.48 | 1.2E−07 |
| GPL | Phosphatidylcholines | −0.35 | 7.5E−10 | −0.33 | 4.6E−07 |
| | Lysophosphatidylcholines | −0.33 | 3.4E−09 | −0.30 | 2.2E−09 |
| | 1-ether,2-acylglycerophosphocholines | −0.28 | 7.1E−09 | −0.26 | 2.8E−06 |
| | 1-alkyl,2-acylglycerophosphocholine | −0.26 | 4.1E−08 | −0.20 | 5.1E−06 |
| | O_Plasmanyles | | | | |
| | 1-(1Z-alkenyl),2- | −0.34 | 1.2E−08 | −0.23 | 4.3E−06 |
| | acylglycerophosphocholines | | | | |
| | P_plasmenyles | | | | |
| | 1-alkyl,2- | −0.59 | 2.5E−06 | −0.45 | 1.8E−04 |
| | acylglycerophosphoethanolamine | | | | |
| | O_Plasmanyles | | | | |
| | 1-(1Z-alkenyl),2- | −0.63 | 1.9E−08 | −0.53 | 2.0E−06 |
| | acylglycerophosphoethanolamine | | | | |
| | P_plasmenyles | | | | |
| | Monoalkylglycerophosphocholine | −0.27 | 9.4E−06 | −0.27 | 1.5E−05 |
| | O_Plasmanyles | | | | |
| | 1-Monoetherglycerophosphocholine | −0.31 | 1.6E−06 | −0.36 | 2.3E−06 |
| | P_plasmenyles | | | | |
| | Monoalkylglycerophosphoethanolamine | −0.65 | 6.4E−08 | −0.41 | 1.9E−05 |
| | O_Plasmanyles | | | | |
| | 1- | −0.52 | 4.4E−09 | −0.35 | 2.1E−05 |
| | Monoetherglycerophosphoethanolamine | | | | |
| | P_plasmenyles | | | | |
| | 1-Monoacylglycerophosphocholine | −0.36 | 3.3E−09 | −0.30 | 5.2E−09 |
| | Diacylglycerophosphocholine | −0.35 | 1.4E−09 | −0.32 | 6.7E−07 |
| | 2-Monoacylglycerophosphocholine | −0.33 | 3.5E−09 | −0.31 | 1.5E−09 |
| | 1-Monoetherglycerophosphocholine | −0.30 | 1.3E−06 | −0.25 | 2.2E−06 |
| | Phosphatidylethanolamines | −0.56 | 4.3E−10 | −0.63 | 2.0E−08 |
| | Monoacylglycerophosphocholine | −0.35 | 3.5E−09 | −0.31 | 2.4E−09 |
| | Lysophosphatidylethanolamines | −0.49 | 2.2E−07 | −0.48 | 4.4E−08 |
| | 1-ether,2- | −0.74 | 3.0E−09 | −0.59 | 6.0E−07 |
| | acylglycerophosphoethanolamine | | | | |
| | 1- | −0.48 | 1.2E−06 | −0.52 | 1.8E−07 |
| | Monoacylglycerophosphoethanolamine | | | | |
| | 2- | −0.50 | 4.6E−07 | −0.44 | 4.2E−08 |
| | Monoacylglycerophosphoethanolamine | | | | |
| | Monoacylglycerophosphoethanolamine | −0.50 | 4.6E−07 | −0.56 | 7.5E−08 |
| | Diacylglycerophosphoethanolamine | −0.51 | 1.6E−07 | −0.64 | 1.7E−06 |
| | 1- | −0.56 | 3.1E−09 | −0.37 | 1.8E−05 |
| | Monoetherglycerophosphoethanolamine | | | | |
| | Phosphatidylinositols | −0.21 | 6.9E−05 | −0.23 | 5.2E−04 |
| | Lysophosphatidylinositols | −0.47 | 1.7E−07 | −0.33 | 8.7E−07 |
| | Phosphatidylcholine-DHA (22:6) | −0.23 | 2.0E−07 | −0.28 | 6.9E−06 |
| | Phosphatidylethanolamine-DHA (22:6) | −0.58 | 1.3E−10 | −0.71 | 1.6E−08 |
| | PPhosphatidylethanolamine- | −0.48 | 7.1E−09 | −0.44 | 3.2E−07 |
| | Arachidonic Acid (20:4) | | | | |
| | Phosphatidylcholine-Arachidonic | −0.33 | 2.3E−09 | −0.31 | 3.5E−07 |
| | Acid (20:4) | | | | |
| SL | Ceramides | −0.35 | 1.2E−06 | −0.36 | 3.1E−06 |
| | Sphingomyelins | −0.29 | 2.3E−09 | −0.27 | 3.5E−06 |
| | Monohexosylceramides | −0.39 | 9.8E−08 | −0.30 | 1.2E−05 |
| | Free Sphingoid bases | −0.22 | 1.2E−02 | −0.28 | 9.8E−04 |

Legend: FA = Fatty acids, Ox. FA = Oxidized fatty acids, AC = Acylcarnitines, GL = Glycerolipids, BA = Bile acids, ST = Sterols, GPL = Glycerophospholipids, SL = Sphingolipids, FC = log2 Robust fold change (Log2), P = value Wilcoxon signed-rank. Underlined P value indicates significance ≤ 0.05

In addition, glycoscale test was performed on the same samples as described above using profiling by nuclear magnetic resonance spectroscopy (NMR). Liposcale allows determination of both the basic lipid profile consisting of total cholesterol, LDL-c, HDL-c, non-HDL and triglycerides, and a more advanced lipoprotein profile including lipid composition, particle size (Z) and concentration (P) of the major lipoprotein classes (VLDL, LDL, IDL and HDL). Comprehensive characterization of the blood lipoprotein profile is addressing cardiovascular disease (CVD) risk. This comprehensive characterization of the lipoprotein profile facilitates the detection of individuals at increased risk of CVD. Glyco scale uses the same technique to determine the presence of glycosylated proteins in serum, which is an indicator of systemic inflammatory processes. More specifically, it determines N-acetlyglucosamine and N-acetylgalactosamine bound to protein (Glyc-A), N-acetylneuraminic acid bound to protein (Glyc-B), and any of the three acetyl groups not bound to protein (Glyc-F). From these three peaks, it distinguishes their area (associated with concentration) and the ratio height/width (describing the peak shape). Considering the extent of the dataset, Table 9 only presents the results for lipid species regrouped in 63 lipid class from the 2.4 mg dose group in terms of Log 2 fold change (FC) and statistical significance (P) at week 24 and week 48 compared to baseline. Pemvidutide treatment reduced the levels of cholesterol and TG concentrations. Moreover, it diminished the HDL-TG/HDL-C ratio as well as the total amount of VLDLs and LDLs. In addition, pemvidutide promotes an increment in the size of VLDL particles. All these changes suggest a diminished risk of cardiovascular events and a reduction in the inflammatory profile. Some of these outcomes have also been identified in other intervention studies on obesity, including some testing the effect of different bariatric surgeries' Similar trends were observed for the 1.2 mg and 1.8 mg doses while no significant changes were observed in the placebo group.

TABLE 9

| | 2.4 mg W 24 | | 2.4 mg W 24 | |
|---|---|---|---|---|
| | vs. Baseline | | vs. Baseline | |
| | log2 | Wilcoxon | log2 | Wilcoxon |
| | (robust | signed- | (robust | signed- |
| | fold | rank test | fold | rank test |
| Parameters | change) | (p) | change) | (p) |
| VLDL-C (mg/dL) | −0.4776 | 2.02E−06 | −0.8218 | 7.23E−08 |
| IDL-C (mg/dL) | −0.1634 | 3.71E−03 | −0.2356 | 3.45E−03 |
| LDL-C (mg/dL) | −0.0761 | 8.34E−03 | −0.0289 | 8.32E−02 |
| HDL-C (mg/dL) | −0.1125 | 6.03E−05 | −0.0567 | 6.66E−03 |
| VLDL-TG (mg/dL) | −0.3547 | 3.56E−07 | −0.5696 | 6.11E−08 |
| IDL-TG (mg/dL) | −0.13 | 1.35E−05 | −0.2289 | 1.84E−05 |
| LDL-TG (mg/dL) | −0.1603 | 1.90E−04 | −0.182 | 4.18E−04 |
| HDL-TG (mg/dL) | −0.2751 | 5.42E−06 | −0.3089 | 1.55E−06 |
| VLDL-P (nmol/L) | −0.3575 | 2.69E−07 | −0.6281 | 4.54E−08 |
| Large VLDL-P (nmol/L) | −0.3812 | 4.17E−07 | −0.5539 | 3.09E−08 |
| Medium VLDL-P (nmol/L) | −0.2906 | 3.72E−04 | −0.5539 | 2.72E−06 |
| Small VLDL-P (nmol/L) | −0.3791 | 1.40E−07 | −0.6647 | 5.16E−08 |
| LDL-P (nmol/L) | −0.115 | 2.87E−04 | −0.0697 | 3.20E−03 |
| Large LDL-P (nmol/L) | −0.0075 | 7.26E−01 | 0.02782 | 2.09E−01 |
| Medium LDL-P (nmol/L) | −0.0815 | 3.62E−03 | −0.0581 | 3.55E−02 |
| Small LDL-P (nmol/L) | −0.1283 | 3.59E−05 | −0.0788 | 4.42E−04 |
| HDL-P (μmol/L) | −0.2006 | 3.56E−07 | −0.1552 | 9.56E−06 |
| Large HDL-P (μmol/L) | 0.01531 | 7.83E−01 | −0.0474 | 9.95E−03 |
| Medium HDL-P (μmol/L) | −0.0343 | 1.71E−01 | −0.0054 | 4.17E−01 |
| Small HDL-P (μmol/L) | −0.2993 | 1.52E−07 | −0.2705 | 1.87E−06 |
| VLDL-Z (nm) | 0.00503 | 5.39E−04 | 0.00482 | 9.73E−03 |
| LDL-Z (nm) | 0.00643 | 1.20E−04 | 0.00652 | 3.22E−04 |
| HDL-Z (nm) | 0.00898 | 4.17E−07 | 0.00891 | 1.35E−05 |
| Non-HDL-P (nmol/L) | −0.1288 | 2.94E−05 | −0.0799 | 2.95E−04 |
| Total TG in | −0.2851 | 1.95E−07 | −0.4941 | 1.10E−07 |
| lipoproteins (mg/dL) | | | | |
| Total cholesterol | −0.1126 | 9.04E−09 | −0.1061 | 1.61E−06 |
| in lipoproteins (mg/dL) | | | | |
| Non-HDL Cholesterol | −0.1034 | 2.43E−06 | −0.1043 | 2.66E−05 |
| (mg/dL) | | | | |
| Remnant Cholesterol | −0.3722 | 3.51E−06 | −0.5999 | 3.70E−07 |
| (mg/dL) | | | | |
| Total-P/HDL-P | 0.00253 | 3.69E−02 | 0.00079 | 1.19E−01 |
| LDL-P/HDL-P | 0.07307 | 2.15E−02 | 0.02528 | 5.47E−02 |
| HDL_TG/HDL_C | −0.1194 | 1.90E−03 | −0.2843 | 7.77E−05 |
| VLDL_TG/VLDL_C | 0.1187 | 5.54E−03 | 0.17113 | 1.84E−04 |
| IDL_TG/IDL_C | 0.02957 | 3.96E−01 | 0.02661 | 9.91E−01 |
| Glyc-A (μmol/L) | −0.108 | 2.75E−05 | −0.1739 | 2.93E−06 |
| Glyc-F (μmol/L) | −0.2113 | 1.15E−06 | −0.2885 | 1.01E−07 |
| Glyc-B (μmol/L) | 0.01776 | 9.91E−01 | −0.0145 | 5.09E−01 |
| H/W Glyc-A | −0.035 | 3.49E−02 | −0.0504 | 2.83E−03 |
| H/W Glyc-B | 0.01408 | 8.53E−01 | −0.0122 | 4.35E−01 |

P = Particle number, Z = Particle size.
H/W = ratio height/width,
Underlined P value indicates significance ≤ 0.05

Other advantages of the reagents and methods of using the same are also provided herein, as would be understood by those of ordinary skill in the art. While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A method for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or a peptide product that is at least 80% identical to a peptide of SEQ ID NO: 1, and wherein the human being in need thereof has a co-morbidity selected from one or more of cardiovascular disease, hypertension, dyslipidemia, dysglycemia, and obstructive sleep apnea.

2. The method of claim 1, wherein the treatment further reduces serum lipids.

3. A method for treatment of chronic weight management in a human being in need thereof comprising, administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1, and wherein the treatment further reduces serum lipids.

4. The method of claim 3, wherein the human being in need thereof has a co-morbidity selected from one or more of cardiovascular disease, hypertension, dyslipidemia, dysglycemia, and obstructive sleep apnea.

5. The method of any preceding claim, wherein the peptide product is at least 85%, at least 90%, at least 95%, at least 96%, at least 97%, at least 98% or at least 99% identical to a peptide of SEQ ID NO: 1.

6. The method of any preceding claim, wherein the peptide product is 100% identical to a peptide of SEQ ID NO: 1.

7. The method of any preceding claim, wherein the pharmaceutical dosage formulation is administered subcutaneously or by parenteral injection.

8. The method of any preceding claim, wherein the pharmaceutical dosage is an aqueous formulation comprising one or more of polysorbate 20, arginine, or mannitol.

9. The method of any preceding claim, wherein the pharmaceutical dosage formulation comprises about 0.025-0.5% (w/w) polysorbate 20, about 0.2-0.5% (w/w) arginine, and about 3-6% (w/w) mannitol in water (pH 7.7±0.1).

10. The method of any preceding claim, wherein the pharmaceutical dosage formulation comprises about 0.2% (w/w) polysorbate 20, about 0.348% (w/w) arginine, about 4.260% (w/w) mannitol in water (pH 7.7±0.1).

11. The method of any preceding claim, wherein the human being is obese or overweight.

12. The method of any preceding claim, wherein the human being in need thereof has a body mass index (BMI kg/m²) of at least 25.

13. The method of any preceding claim, wherein the human being in need thereof has a body mass index (BMI kg/m²) of at least 30.

14. The method of any preceding claim, wherein the human being in need thereof has a body mass index (BMI kg/m²) of at least 35.

15. The method of any preceding claim, wherein the human being in need thereof has a body mass index (BMI kg/m²) of at least 40.

16. The method of any preceding claim, wherein the treatment maintains weight loss.

17. The method of any preceding claim, wherein the treatment reduces relative body weight by at least about 10%, by at least about 15% or by at least about 20%.

18. The method of any preceding claim, wherein the treatment reduces lean body mass by less than about 25%.

19. The method of any preceding claim, wherein the human being in need thereof has an HbA1C value of about 5.7% to 6.4%.

20. The method of claim 1-14, wherein the human being in need thereof has an HbA1C value of about 4.0% to 5.6%.

21. The method of any preceding claim, wherein the pharmaceutical dosage formulation comprises about 1.2 mg of the peptide product.

22. The method of any preceding claim, wherein the pharmaceutical dosage formulation comprises about 1.8 mg of the peptide product.

23. The method of any preceding claim, wherein the pharmaceutical dosage formulation comprises about 2.4 mg of the peptide product.

24. The method of any preceding claim, wherein the pharmaceutical dosage formulation comprises (2.4 mg/ml; 0.24% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

25. The method of any preceding claim, wherein the pharmaceutical dosage formulation comprises (1.8 mg/ml; 0.18% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

26. The method of any preceding claim, wherein the pharmaceutical dosage formulation comprises (1.2 mg/ml; 0.12% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

27. The method of any preceding claim, wherein the treatment reduces one or more serum lipids selected from serum triglycerides, total serum cholesterol, serum LDL, or serum VLDL.

28. The method of any preceding claim, wherein the treatment reduces serum triglycerides by at least about 30% after 48 weeks.

29. The method of any preceding claim, wherein the treatment reduces total serum cholesterol by at least about 10% after 48 weeks.

30. The method of any preceding claim, wherein the treatment reduces total serum cholesterol by at least about 15% after 48 weeks.

31. The method of any preceding claim, wherein the treatment reduces serum LDL by at least about 10% after 48 weeks.

32. The method of any preceding claim, wherein the treatment reduces serum VLDL by at least about 25% after 48 weeks.

33. The method of any preceding claim, wherein the treatment reduces serum triglycerides by at least about 30% after 48 weeks in human being in need thereof with >150 mg/dL triglycerides at baseline.

34. The method of any preceding claim, wherein the treatment reduces serum triglycerides by at least about 50% after 48 weeks in human being in need thereof with >150 mg/dL triglycerides at baseline.

35. The method of any preceding claim, wherein the treatment reduces serum cholesterol by at least about 15% after 48 weeks in human being in need thereof with >200 mg/dL cholesterol at baseline.

36. The method of any preceding claim, wherein the treatment reduces serum cholesterol by at least about 20% after 48 weeks in human being in need thereof with >200 mg/dL cholesterol at baseline.

37. The method of any preceding claim, wherein the treatment reduces serum LDL by at least about 15% after 48 weeks in human being in need thereof with >130 mg/dL LDL at baseline.

38. The method of any preceding claim, wherein the treatment reduces serum LDL by at least about 20% after 48 weeks in human being in need thereof with >130 mg/dL LDL at baseline.

39. The method of any preceding claim, wherein a steady state therapeutic dose is achieved after a dose escalating phase having a duration of from two to four weeks.

40. A method for treatment of reducing serum lipids in a human being in need thereof, comprising administering to the human being a once weekly therapeutically effective amount of a pharmaceutical dosage formulation comprising at least 1.2 mg and up to 2.4 mg of a peptide product according to SEQ ID NO: 1, or peptide product that is at least 80% identical to a peptide of SEQ ID NO: 1.

41. The method of claim 40, wherein the human being in need thereof has a co-morbidity selected from one or more of obesity, cardiovascular disease, hypertension, dyslipidemia, dysglycemia, type 2 diabetes and obstructive sleep apnea.

42. The method of claim 40 or 41, wherein the peptide product is at least 85%, at least 90%, at least 95%, at least 96%, at least 97%, at least 98% or at least 99% identical to a peptide of SEQ ID NO: 1.

43. The method of claim 40 or 41, wherein the peptide product is 100% identical to a peptide of SEQ ID NO: 1.

44. The method of claim 40-43, wherein the pharmaceutical dosage formulation is administered subcutaneously or by parenteral injection.

45. The method of claim 40-44, wherein the pharmaceutical dosage is an aqueous formulation comprising one or more of polysorbate 20, arginine, or mannitol.

46. The method of any claim 40-45, wherein the pharmaceutical dosage formulation comprises about 0.025-0.5% (w/w) polysorbate 20, about 0.2-0.5% (w/w) arginine, and about 3-6% (w/w) mannitol in water (pH 7.7±0.1).

47. The method of any claim 40-46, wherein the pharmaceutical dosage formulation comprises about 0.2% (w/w) polysorbate 20, about 0.348% (w/w) arginine, about 4.260% (w/w) mannitol in water (pH 7.7±0.1).

48. The method of any claim 40-47, wherein the human being is obese or overweight.

49. The method of any claim 40-48, wherein the human being in need thereof has a body mass index (BMI kg/m²) of at least 25.

50. The method of any claim 40-49, wherein the human being in need thereof has a body mass index (BMI kg/m²) of at least 30.

51. The method of any claim 40-50, wherein the human being in need thereof has a body mass index (BMI kg/m²) of at least 35.

52. The method of any claim 40-51, wherein the human being in need thereof has a body mass index (BMI kg/m²) of at least 40.

53. The method of any claim 40-52, wherein the treatment maintains weight loss.

54. The method of any claim 40-53, wherein the treatment reduces relative body weight by about at least 10%, by at least 15% or by at least 20%.

55. The method of any claim 40-54, wherein the treatment reduces lean body mass by less than about 25%.

56. The method of any claim 40-55, wherein the human being in need thereof has an HbA1C value of about 5.7% to 6.4%.

57. The method of claim 40-56, wherein the human being in need thereof has an HbA1C value of about 4.0% to 5.6%.

58. The method of claim 40-57, wherein the pharmaceutical dosage formulation comprises about 1.2 mg of the peptide product.

59. The method of claim 40-58, wherein the pharmaceutical dosage formulation comprises about 1.8 mg of the peptide product.

60. The method of claim 40-59, wherein the pharmaceutical dosage formulation comprises about 2.4 mg of the peptide product.

61. The method of claim 40-60, wherein the pharmaceutical dosage formulation comprises (2.4 mg/ml; 0.24% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

62. The method of claim 40-61, wherein the pharmaceutical dosage formulation comprises (1.8 mg/ml; 0.18% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

63. The method of claim 40-62, wherein the pharmaceutical dosage formulation comprises (1.2 mg/ml; 0.12% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

64. The method of claim 40-63, wherein the treatment reduces one or more of serum triglycerides, total serum cholesterol, serum LDL, or serum VLDL.

65. The method of claim 40-64, wherein the treatment reduces serum triglycerides by at least about 20% after 48 weeks.

66. The method of claim 40-65, wherein the treatment reduces serum triglycerides by at least about 30% after 48 weeks.

67. The method of claim 40-66, wherein the treatment reduces total serum cholesterol by at least about 10% after 48 weeks.

68. The method of claim 40-67, wherein the treatment reduces total serum cholesterol by at least about 15% after 48 weeks.

69. The method of claim 40-68, wherein the treatment reduces serum LDL by at least about 10% after 48 weeks.

70. The method of claim 40-69, wherein the treatment reduces serum LDL by at least about 20% after 48 weeks.

71. The method of claim 40-70, wherein the treatment reduces serum VLDL by at least about 25% after 48 weeks.

72. The method of claim 40-71, wherein the treatment reduces serum triglycerides by at least about 30% after 48 weeks in human being in need thereof with >150 mg/dL triglycerides at baseline.

73. The method of claim 40-72, wherein the treatment reduces serum triglycerides by at least about 50% after 48 weeks in human being in need thereof with >150 mg/dL triglycerides at baseline.

74. The method of claim 40-73, wherein the treatment reduces serum cholesterol by at least about 15% after 48 weeks in human being in need thereof with >200 mg/dL cholesterol at baseline.

75. The method of claim 40-74, wherein the treatment reduces serum cholesterol by at least about 20% after 48 weeks in human being in need thereof with >200 mg/dL cholesterol at baseline.

76. The method of claim 40-75, wherein the treatment reduces serum LDL by at least about 15% after 48 weeks in human being in need thereof with >130 mg/dL LDL at baseline.

77. The method of claim 40-76, wherein the treatment reduces serum LDL by at least about 20% after 48 weeks in human being in need thereof with >130 mg/dL LDL at baseline.

78. The method of claim 40-77, wherein a steady state therapeutic dose is achieved after a dose escalating phase having a duration of from two to four weeks.
